SEQUENCE LISTING (19 sequences)

SEQ ID NO: 1 - protein, 14 aa, Rattus rattus
SAALRQQMEALNGK

SEQ ID NO: 2 - protein, 14 aa, Homo sapiens
VASLRQQVEALQGQ

SEQ ID NO: 3 - protein, 14 aa, Bos taurus
VNALRQRVGILEGQ

SEQ ID NO: 4 - protein, 14 aa, Mus musculus
SAALRQQMEALKGK

SEQ ID NO: 5 - protein, 14 aa, Sus barbatus
ITALRQQVETLQGQ

SEQ ID NO: 6 - protein, 21 aa, Rattus rattus
SAALRQQMEALNGKLQRLEAA

SEQ ID NO: 7 - protein, 21 aa, Homo sapiens
VASLRQQVEALQGQVQHLQAA

SEQ ID NO: 8 - protein, 21 aa, Bos taurus
VNALRQRVGILEGQLQRLQNA

SEQ ID NO: 9 - protein, 21 aa, Mus musculus
SAALRQQMEALKGKLQRLEVA

SEQ ID NO: 10 - protein, 21 aa, Sus barbatus
ITALRQQVETLQGQVQRLQKA

SEQ ID NO: 11 - protein, 1487 aa, Homo sapiens
MIRLGAPQSLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 12 - DNA, 31001 bp, Homo sapiens
atgattcgcctcggggctccccagtcgctggtgctgctgacgctgctcgtcgccgctgtccttcggtgtcagggccaggatgtccgtaagtcttcccccgccgctgcctgcctgcctgctttccatgcgtccctcagcatccttctccccggcccgctccagctctggagcccgcggctccgggctaaaacggctcccggggtcgtagcgcgccgacttaggcacaggacacgcagaagttcaccaagaagagttctgccaatcaagactctgtcccagggtcctcggtgcccatcgcagttgcaagtatttgcaggtccctacgttgcgctagaatactgaacttgcaaagtgttggctcggagaagtttgcgcacagatataaatgggctcttttccaccagctttgataattaggcgcacatgcacacagctcgcctcttcgaagcacttcgagttcagcaaaaacagatctcaactcatcgaacttaggtgaagtaggaaagagagagcgcgacggggagcaagcaaacgccaaagggttgacttcacagcctgtccaaggcttggtggctggtgggctcaaagcagagttagacaaaggggactaacactctgacactggtgggctgaaatcccaggccacaaagaacggcttccgataggccctctgagacctcagcgcctctttagggtaccctccccctcccagctggccctggagcaaggtgcagccctagcgctcatctcgacttccctccgtccgcctgcgcctctcttctgataaagggtacagaaacttccagtaggagaggccatctgaaagacgataacattccaaccagaccgtgcttttcaaatgcccccgaaaatagcgcccccttccccgcggtcttaccccattccccgccgccccgaggtactacaatgagttacttttctaaattctggaactcaccgagccaggctgcgtggtgtgtgtgtgtgtgcgtgtgtgtgtgtgtatgtgtgtgtcagggaaggggagcaggctggtcgattgctacggttgctacaactatttcaaccggtatagttagagatggctcttgtagtcgggtccaaatgctgttcggactgcacctttctacccctcctttggtaaggtccactgtctgggattatattcaggacaaacgaagcctggaaagtgtattaggtagagaggatttttttttccacgtgtttgggcacgtttccgacggctgggattccagccctgtctttgtatgttacagattgtaaatcaatcgcagagggaaactcttcggcgggggaaataaaagttctctgccttcgaggctctgtgggccctctcctgccaccaggctgtttccagggatagcgtggaaggcggcgggctcaggcgggctttccggtcattagcgcagcgggggcagggctggagcctgcggcgcagctgcgaggagccgggagcaggagactctggccgggtcacccggtagtgcgctaagctggaggcgcgctcctgggcatttgaggaatacagcgtgactatacgtggcctggactcagactgactatatttttgtactaaatttacaagcacacgcccacaaagctgtcttcttgactgacccctgccttagtgagcaatggaattagctgggtggctttaaaataattctcaaattctccatccggtattagggtcgcttgcttaattaggcggtagaggtctctcatcgccgcatctttcctgggagggagtgattccacagcttctccggcccaaaccttccagtcgctcctcctcccagagggagtgtgattctgcatccgagaggctgattttgcgccctggagcatcccaccttttataacttcccccgcctggggtcagcggacccaaaggtgtgacgtggggaaatgcgcagtctgcgtggacgtcaggaatgtcagacacctagagctcggccacacccctcctctccatctttccacgagtttgagaaacttactggcggcggcgtctttgaccctcatctgcatttcagagccctcgcctccgaaagtgcccctggctcaggggagagatctcaatcctcctttgtgaggcttgtttgcattgggagattggcagcgatggcttccagatggggctgaaacgctgcccgtatttatttaaactggttcctcgcagagacctgtgaatcgggctctgtgtgcgctcgagaaaagccccattcatgagagacgaggtccagtgggttctctcgtactcccagaccccctctcccacaatgcccccctgtgcccgcccgccgccacctctcggctccagccctgcgcagagcggcggtgaagcaaaacagttccccgaaagaggtagctttttaattggcttgccacaaagaatcacttatacggccctgcggtaatgaggggaaccggatcaggcgcgccgggatgctatcggcagccgttttggagcagcaattatggtggtgctgggctcctccgtccacacctaggggatccggttacggcgctggctcctttctggggcagtcatttaatcccacttttcactctcccggtgtctgtgagcgagccgtgtccagagccgcagccacagagtcactcagcggctcttacacccagcgcagcctggccccgcccctgcgccggcgcttcccgggccgcccttccccgggaaatctgatccgcacggggagtggcccctctcctagcatttccccctctcctccctgggtcctcatgggcgagggtgggctctcctgtagtctgggctggagcgcattaaccgatgccccctctcccacaccttcctcaccgcctgcattccactgctccagctattttaacggcgggtgtgtccccgcaacttctgtattttccctggaatccctcaccctcctgtgattatcttgcccaaaggctaggcggatttcttctagtgggaaagtaaaaaggaacgtttatctttggattttcactctctttaaagagcagtgggcaggctcgtttctttctccgcctctgggtttgtggctctttcctattattcatcccctgctgctgctattgccttggggattttgatgagaaaaacacgctgggcgctccctacgacgtggtgcggctctacagcccttggctgctaaggagcgctcttgtcagcacaggtttcatttgcagcatgaattccagacggcagggcgctggtggaggagactagtccctgctattcttcctctgcagtcttggaggaggccaggcctggactggcaatcttagccctagccaggtattcaacgacccctgctccccaaactggggtgctgttttcagatggaggcagggcctctccaggcagggctacaggtggaggtcagcactgggggcgctttggctccactggcctcctaagcagtttattagcctgcccaagccccaagtgtattgtttgaatgggtctatccccctccccaaattggtcctaattctaatatggttcaaagaatgagacaagatcctaattctaatagctcgtcttttcacccccctttcttatatacctatttttggagcctcactgcttatagattccaatttttgtaggtagaattttctacattccctctgaatgttagttgtcagttgtatttagctaatcccataattcccagaggaaggcagaagaaagaagacttctctgctcctgggctggtggaagggaggtctcgccatttttctgtctcctttctttttatagtcccagaattcctattcagaatatcttgtctcctcccttccgctcaccctccaactccctccacccactccatcacctggtctcccccgtattaggtgggtaaagagaatatagtatagtaaccccccaccttcattgctgggtcaagattttcactggtgaatagacaacatggtgcaaggtgcataataaatatttgttgaatacatggaaaaatcaatgatgttttaggaaaataatttttaagttctatatgtccaggtggccccagcctacattcttcagcatttgaattctgtcaagttgactgcaacctctctctttttctctctggctccccaccccctccttcccttggctctctgcttctccctccccacccttggtgcagaggaggctggcagctgtgtgcaggatgggcagaggtataatgataaggatgtgtggaagccggagccctgccggatctgtgtctgtgacactgggactgtcctctgcgacgacataatctgtgaagacgtgaaagactgcctcagccctgagatccccttcggagagtgctgccccatctgcccaactgacctcgccactgccagtggttgtaatttatttatttcctgttcaacataaataaattacttgcaagcactgcaaacacgctcccatagatgctggtcgtctctgcaaagcagaggggctagttatccatgggacctggtagctggggtagaaaaggaaaaggccacttctcacttgcaggttgaaactgagtgaatgagcctgagacactagaggggtccttctttgcccaacatctccaaaaacatttgcttccaagacacatgaaggacagatgtaattctacaaaaaaaaaaaaaaaaaatcctctctgaaatcatctctgcaaattactagagccactatggagatcaaatgctctgtcttggccaatccacgaattaattcctcctctgccaccgataccttgtcttctccttagaagacttctatgtatgtggtcttcagtgtggagaaagctctgccagctagtggggagactgcaggggcagaggctccctctttgagttatggaacattggtggtagtttcctctctgctattacctctcttggagttgaccattaattcagaagcaaaataataagagagggaagggctaggctttgggagttctagtggggacgggtggagacagagccccatgtatctgcactgtagtgggtggttataaactcccagttagatccagtgctggtggatgatatatgtgcaggtgaccccttccccagcattcaatacaagatgtcctatctcccctgcagagtgagtggggacgcttgtgtaggttttttgggtagctcttgctgtccccttcctgctgaagtagagaaggccgtggcaagggaagtgagaagctgcctttccttaacacttcaccaacactggctccctaatgtgcacattcccagatcctttctgaggggcccgtgtgagtgaagtgttgattgcctttactattttgctgctactgtgaaggagaggttattgactggggtggcacaggctatgatgctccgatgctcttcataactcatatgccttgctgtttttgtgtttttatttgtgcttgcttcaaggagacccagctctaatgtaagacctttctaagtacctaactcttcctctgggagggcttggggttcgggaacggctccctacctgtggggggaagagagactgaatctgtgctttccttcttgtggctgattagatcttgagctcttcattgcctttttgtgctgcccttgctcctttcttttgcatgctgcctgctttttgaataacaaagcctgggtcacctccatatcctcatgggacctcagcaaccccaggccacagtggccctaacaccccaacagaggggttcagtggagtcacaggaacgtgccgccttccttgattgtgtccttttacttgtttgatctaatgagtgagtgtttgagtgacaagaataggtatttttccatctcaagattcttaccttcttcttctctatattttttccttgcagggcaaccaggaccaaaggtaagggctttcttctttttcttttttcatatttttttggctttatattttctgcttcaaaagcaatgctatgttaatccagtctgtgattttttagacatcagaagatatctgtttcagagggtacctcaacacaggggctgctggcagggttttagactaggggcttagtgggcttactcggcttaatcctgtgaatgtttcatgtttcagggacagaaaggagaacctggagacatcaaggatgtaagtgcaaattattctcacccggtattcgacgtcgtcgtctaaatgggtcatttccttgtgctctcctctaacttaccatcctgtggggctctctctcacagattgtaggacccaaaggacctcctgggcctcaggtaagagagggagaaaatctctttctccgtcccttcctcgctgcgcaagttactgatctgtaactcctggccttgctgtcatcttaccatgttcttcaccttcagggacctgcaggggaacaaggacccagaggggatcgtggtgacaaaggtgaaaaagtgagtaaaaagcaatgctgcttgaccctggtggacttcccaggtcccccaaggccccaccatgtgtttaagggcctggtcacctcttaaagagcagccaagggacagatggctcttggagaaacactgcttcccattgatgcctttttctctttatgccaagggtgcccctggacctcgtggcagagatggagaacctgggacccctggaaatcctggcccccctggtcctcccggcccccctggtccccctggtcttggtggagtaagtatccttacttcccattccttcaggctgtccctccagaaatgtggcttttaaattgctgcttgcacttacctggctggctcccagggctgccagcagtgtgtacatagcctgtccatgggctttgctcaggcctgtaatttagaagagtcacatattaggcatgagactgtggtgctaagggctggcttttttcactaactgggattctataaagaaagtcctcagttacctggcttcctggcatctgtaccacgtagttgatgctggggggtgggtgtaagggataggaggaaggatgactgggcacttgtatttccctggaagacgagtgaccactgtccttggaagacatttatccttggttcttgccaagtacattccaagcaactattcactctcatgaaagagctccactgagtgaaggtgtgtggctaaagtcaattctggaatcaaaccaatcaacaaattatatgattgcctagtttttgcaggtttgctattttgatgtttgctgtattttaaattcttaaactcaaatgggatcacagatgcctactacatctcttgctgaaatattccaagactgttgattttagtcttttgctgggcactaaagtctaaagaataaagaacacccttagaaggtttggttatgtttctccatatacgttaaataacatctgtcatattttagagcataaaaataattttataaaatgaaatgcaaggaactgatacttcctcaaataacactttcccttccagtgaaatgattttgccactgtcatctaataatccattcccaaaattactttccaggcatcagtggtaattctgatcaatgataggttagtctccaacaatcagagtttatctcagtagagttctttgtatccatatagagacttacctagccaagtagggaagacctagtgcctttcaacctcctaacgttgttgggtttctttgcagaactttgctgcccagatggctggaggatttgatgaaaaggctggtggcgcccagttgggagtaatgcaaggaccaatggtaagaaaagacactagttctttgcagccaaaatggcaggaggtggcccttagcagagccagagagtctgacaacctctgctttacagataattgcttagagtggctctcctccgtagttatgtaacctcccattcagctagcccaaagcatttggtttttaatggcaatggatgccacttttaatgatgcgctggagtgactaagaagaatgaagatgggagatgcatataggctgatctgttagaaggccagttgctattgctcttggaatgagaactgaagaatgcagacagcagctactgttctccagcatccacagacttccagcaggccctctcagcccgcagctctgacttggcacatgctaaatgaaactcagcctttagtaaacatggctgctgtccaggagaaagcaaggccagcttttctgtccaaatggtgcctataaataaaaatagagtgttgcgtggggagtgggaaatgagagggagcagccactctaggccccttgcccacagagtaacttcttgtcctttgcccgggctgttggctgggagaagatggcacactggaggccactgaggaagcatgtgtagtaaacccctcattttctgttccgatgcagggccccatgggacctcgaggacctccaggccctgcaggtgctcctgtaagtatctgcaagtctttttgcctccatcgtgtcgcagatgattcccaagcactatgatgttttagcagtttatagggattgacctggtatcctcattttactttttaggggcctcaaggatttcaaggcaatcctggtgaacctggtgaacctggtgtctctgtgagtaccagcacggccctgtcccttctctgggggagcctctaatgatagaccactaggacgcagctgctgtccctcccagctctgcccagctctttcccacagtcggtggccccaaggaaattcggatgtcacttcctagctgtggaggaactctcacagacagcccaatgtggcaaggaccaccagggactctgtcctaacagcccctttggggtcaccccagcctgtgctatctgctgcaatcccactatgatctctgcacctttgctctgaccttcccatctttcttcttcatagaagaactggcattccaaaactacaatgtcaaagttttgtccattgcttaggtgtcttcccactataaccatctcttaaactatcttcctttgtttgtaagggtcccatgggtccccgtggtcctcctggtccccctggaaagcctggtgatgatgtgagtatacacgagtagacaaatgaggagctgcctcctttgaaagggcctggagagggtgtgtgcttggggagtgacagggaggcacccagggtggaggtatcttgaggagcaagactgggcagtcccaaaccctgacgccatctcctatctatatggccactgtgactgtgctggcaagttccctggggaccgctttggatccaaggggaagacaaataattaaaacatcattagccccaggaagggaaattgagaaatgagagaagggagagaaaaaatacaaggcagaaagatgtagagaaggaaaaacaaagaaagaagcgttcaacaacccagcattatcttaattgtaaatgagttagaaaaagcacagcctgagtcaggatgtctacaaaggatgcaaactgaaatgaagagacaagaattggcactcttgtcgtatttttatgaattcgattagacagtaaaagtctcttgaggttagagagagcacatacagtcagcagaacctaggagaggagagaaaagcctctcaggggaagttggaggctggtgaggacagaggagcttgcccatggcgtatgcatgtgtccaaaagaataaatggtgacccatgaaaggcatccaggcacgtggagtctgaaggaggtgagggagatgagtgagccggtacagaaggcatggagggctggaaggagggaagccctctgggtgcccccactatgctactgcgtctctgaggaagctgggatatctctctctctcccttcagggtgaagctggaaaacctggaaaagctggtgaaaggggtccgcctggtcctcaggtaaacgccaccgttcccagcctcaggcatctttcctagcgtctccctccctgtggccttaaacacagtgcatccagttcaatgaggtcacttctgagatgaaacgccagtagcccctatatttatcacgaccatgtttgtaatttccactcaggctctcatgagggaggctgggcagttgttatttataccactttgcataaaatggggggtacggggaggggtggtcgtggttttacagaaagagctgtccaagtgtggggattcgagacaacgccctggtggcgaagggaactggaggccctcctgcagccagggcagctttccactgttattttactctgtgctctgaacacctccactttggattgcagggtgctcgtggtttcccaggaaccccaggccttcctggtgtcaaaggtcacagagtaagtatcacgggtgagaaggttggaaggaagagatgcctggtgggagagaaaagcactttggggtgcgtgcatttcttccaacttgggtttcccagaagtctgattgaacattttctcttgttccctagggttatccaggcctggacggtgctaagggagaggcgggtgctcctggtgtgaaggtgagaggccagaaagtacaatgggatggggaggagggagacaatgaggagcccctcttcctagccagggagacactgtggagctcagtggaactagctcctcagaacagccttggctgggaacaccagccctacatcctgatgggccaacagcaggcctggagagctcagggcattgtccctcacaggactgaagtttgtgtcagtgcgagctgagatgaccagggcttttggcgtcttccctaggagtttgctggcggccaagaatggggtcccagacactgaccttgtgcatcatttttccagggtgagagtggttccccgggtgagaacggatctccgggcccaatggtaagtatggacaccctccaggaaggtttatccaaagactcttcagactatcagatggctgcaaagagctccctttgtgcaaagttcatattctgtgttgtagatttcatctgattgtgagcaaaaagcaaaatgtattagacagatgatttgttcaagatttcaccaacatttccttaagatagccatgttatcacactaaagatgctcccattttaaaaaattctgttgagtctcaacattttgtcaagctcatctactgcaaggagcaaggtgtgcttgtaacaaaggttcccaataggtagcaacaggaacattcgtgtgttccgcctgtggagaaactgttgggtgtgatctgaagcatcctggctagtcaaggagccagcaccatcaggaggtccttgttttcctgggtgtgggcatcctccctctcctctggtatccgcaaagggcctgcaggtagaaatggtcaccctgagcaccgtaaagccaactcatgcttaggctgtcctggtgtgtgttccagggtcctcagggtcctcgtggcctgcctggtgaaagaggacggactggccctgctggcgctgcggtgagtaattgacaaagccaaacaccaccatttgccgagcactttagagtttacaggtttgtttctcttgaccctcgaaacaaacctgtgaggcatagggagtattgctatcccttaagaattcaccccagggttccatcaaagcttccaggctgagtctcacagtgaaggaggaaggataggaatgggagggtcgatgggtgaaagcatgattctcttaaccagtccagattatcaggtaatcccttcaacaaccaccacccactccctgggcaatccagctggagtttacagacagacttagctggctatagcaccaccgtgctactctctgttcttcctggttgctcaaatgccctagaaaagtggaacaggtgagcatcaactcacagggctctatgctggctgctgctgcgagggatgttatgctatagtaccaggggccaccattccataggcacttcctgtgtttaataccctatagctttacttcatctcatcttcctccatatcctgagaggtggttctattcttctaccccattttacggatgaaaaaaccgagacacagaaaggtgaaactagcttaagataaatggtgccttgcagccttagactctggtggcctctagttaatgtgggaaattaagggtgaggggattggcagctgatggagggtgcagggtgccagacagaggcgtttagctctgatcccttagcaatagagagtccttgtaggcacttggtcaggcgagtgatgcgatgaaagctgtgtttaagaaagattatgctttctgctgatttcatacccccaacacccaagctctgaggcccctcctcacaggtccttgcagggctggccaaaataaagcagcttcactccgttgtgctgctttccagctaatgtgtctgtttggcagaagtttccctcaaaggcagatcagtgaaataagcagaagcctcgacccccctttgtcagccagagctgctgaagtgccttgccccagggtcactttgtgtgaggggattagagagcactggggctgccaagaaacactgccgtttctacagattagcaggacgctggcttgtggcttttagcgaggctcagagctgcggtggccctagtctgcatgggctaaagacaagctccatctcctgtcctttttccctccttcctgggcacagccgccctgcttcttggttctctctgttggttcctgtccgcacggtagttaggctggcagcgtgtgtaggatttggcttagaagattgacaacattgcctttgagcccttctttgctactcctccctctcccctcccatcagactcctctctggagtctgctctgcgaggcctctgctctgtggtatcccagcagccttctcagccttgacttccagaagggggctgtgcagtgtccggggtgtgcaggccccagacacggggtaggctcatggagatccaagtgctgatctagtgtcaaggctggcctggagactgggctgggttggtgtcagcctgctgtggtcatgtgccctcccaagggcctgtatcctctctccagacttgctgcagggagaggtggcagatgtcagcctagttctggcctctcagagcagcatggcagctccctttcactcaggcccaggctgggcctcctgctggctgaccctggggagagggtgctccagagcaccccaaggaacagcttcccgaagcagccaggccagcccagaggggctgtggccaatcctgaagctttatgttcctgctgacattttttctaagttttctcttgctttcctcttaaatgccaatctggagagtctccgttaggagaaatggaccccagccaggaagaagagttgagttgtatttaaaacacgagctccccctaaagcatccttctttagcttctaaggagaggcagagactgacaggcaggactcagcaggtaaaagtacccccctgacctgctcagtcagcctaggcccagctccacccagcctgtggggcccagagtttcggtaaagagttccctgggccttaaggaaccttgagagagcatttgaggggtgccaccacaaacttggcagaaaaaaccctccccctccaagtccagtcctagagaaggagctggcaaccttgccttgctttgtaagcaaaagcctcttagggcttgagtctagatgtagtgtttgagctgtggctggtgccctcccccatcagggagccaatggtagacatcctatgggcatctttgttttccgtaagagcaggctgctcggggatgggccagaggaagaggcaacctggagtcaaccaagaggaggccttaaccaagccttaaccacagaggttaaccaagccttgaaagcgcttccccctgagcaggccaggaagcactgagtccacatggttgcctcgctgtttcatttccttacactcaattctctcagtctttaaatgatcacttggccttgaagttacggatatttggggtctgaactgaagttgaagaaaagaggaaatgatttaagctttgtttaagattaggggccaggtgcggtcgtcacgcctgtaatcccagcaccttgggagcctgaggcgggtggatcacctgaggtcaggagttccagaccagcctggccaacatagcaaaacccagtctctactaaaaataacaataaaaaaattagccaggtgtggtgacacatgcctgtaatcccagttactcaggaggctgaggcagaattgcttgaacttgagaggtggaggttgtagtgagccaagaccgcaccactgcactccagcctggcgacagagccaagctccgtctcaaaaacaacaacaaaaaagattagaagaagcccattactgccttctggccacccactcgcacagacaccaaaactgcagcccacacctcgccatcctcgtgctctgccctgggacaccccaggcacagtgtgtccttcgttttctgtaagggtgggctgggagcagggacggacagggcctgtgggcacctctcatggtcacttccttcttgctcacagggtgcccgaggcaacgatggtcagccaggccccgcaggtcctccggtaagttcatttcatcctcagcaggtcattgttgctgtgctttaagtcccgttaagcagcccaaggcagtctcgagggtgtattgggtgcaaccacagcagcactctgatgtctactggaaagggggaggaaagagaagaagtttgtaaatatcaattgagcatatcgataacaagctttgaagcatgggctcattttcctcagccatcctttcagcagtctttttagaggagggaggtcaaaggagtttctgcttctcaccacagatgtagtcagaaacttgctttgccttctgaagccaggcaaagcttcctggggacgctggcaatggggacaattttcatccaaggccttttagccacaatggatatggagtgaaatcagtacagaggagggaaggagtgtgaggtgtcggggtcgctcgctttggaggccagaactggcattcacctctcttctcatccgcctactctctccagggtcctgtcggtcctgctggtggtcctggcttccctggtgctcctggagccaaggtacgtgccctgttgtccagtcaggaacttctgggtgccgagaagctgtcctttccccgtaacccttgctcattgctccctcaacaaccacctgctcccttctgagaagtagctccccaccaccccacccactggcccctccatccaggcagggcaaaaagccagacactcgcagtctcacctggagggaagtaagacagaagataaaatgtgggagatccagttacaactttggagtggggaaaggtggacagagaagaagacggggatacaccataggcctggcaggggcagaaggccaggagtggcagcacagggaagcaaacctaggggagacccaacagctgagcaagctcggccggtgacgggcatcggagggaactgggcagggaaaagggcacaggcaggagcccctgctccctctgggtttctgctttatttggggtgcctggctcttccaaaccatgttaacggagttctctggaggattactagaggccagtgggaggccagccagttcagggacaggcctcgcagcccaggaaggattccagtgtgaacgtccctgggaatgaataaggagcctccatgtgtcactggcatcaggttgcttttccctcctggggctttccatggcaaccagacagtgtctgaggtccggagccgggtgaaggagacccattgtgaagagggacagcggaaggtgaggggggctgacctttggaaaataataattaccacagtgaagcaggaatgttctgagaagaaacctgaggagctctgccctctctccaggtcagcagccctccccagggactctgccatctagagtgggttgtaattttcaggaaaaaatgaaagtaaaagcacaagccattttgtggggagggggcttgccagaggcgcccgctaaggggaattgggctgtattgagagcagggaggggcagagtccccatgtgcttttgccttggctttctggcttactgagaacagactggggccggagccagggtgtcactgttcacccatcagccagatgggagtgaggtggtgctctgagctgggatgttcagagacttagaagggacctcagctcctcaataaaatagaaaaacaggaggtgggggagagagcggtgtccgtccatcatcccacggtgccaggatggcagggtccccagcccacgcttttctgatggtgtcgatggaacagcaggttgcccattgctgtagtatgtagctgtgccgtggcatgtggaggctcactgtgtagagatgaggtaagcagtagaggaggcaggcgtgggaagtcatcaagtcatcagctcggtcaggcagggagaaaaacggcagcgtgaactgtgtgtgaaccgacatgttcatgtgcagggttgggtgcatgtgcataatttagtgctgtcgttgcagctggaccctgagctattgcccacccactagaggtctgtgtcccctctcttcttcttcatttcatacctccctgtctcttcccagggtgaagccggccccactggtgcccgtggtcctgaaggtgctcaaggtcctcgcggtgaacctggtactcctgggtcccctgggcctgctggtgcctccgtaagtgcagcttctctttggcctgggggggtctggggtctgtggctttggaactcttgactctgtactttgctctgacagttgtgggctccaaccaccaaaccttcattctggcccaatgcctgtcccacctctagatgtattcccttctatcccatcttccccttgaaacacatagtgggaatgtccctgaaatggacagcacctatgccaggtccctggatctggatcctggagggctggaggtggttggggttcattctttgctgcttatttgacaatgtctcccttttcagggtaaccctggaacagatggaattcctggagccaaaggatctgctgtgagtgttgcccgtggactttgctaccccaggagagcccagtcctgcctctcccctctcctgacacccctcccttcttctcatgcccacagggtgctcctggcattgctggtgctcctggcttccctgggccacggggtcctcctggccctcaaggtgcaactggtcctctgggcccgaaaggtcagacggtaagagcccaaagtgacccccaagttccactgacatctctggagtcaaaccccatcacccctctttcccatgctctcctgccctggcctcacagcggcctccatccgagggcatcttgaacaggggttctggggaggggcaggctccctggagagaatctggtgtgaggacctgcctctcttttcaagggtgaacctggtattgctggcttcaaaggtgaacaaggccccaagggagaacctgtgagtatctgcccccaagcccttgtcttctctgctgctgttctatgaggcacagcctcagccccactgacccaccacctccctcctccagggctctatcccccaatctgggtcctttcagattatgcctggaggagacttaactgggctgagaaggcccagatacagcttcagctcccatccttggtttggctagtgtgaacagttggatctttagcccctctcacttccctctgccctgccatggctcgtcctttatgcctggaggagacttaacagggactgagaaggcccagatacagcttcagctcccatccttgggttggctagtgtgaacagttggatctttagcccctctcacttccctctgccctgccatggctcgtcctttatggcctctcgtcctcaagcccccccccagccctgaaacagttgccaaggctacttccttcatactctagatcgaggcttgctccaaggccaggtgaaggctcactctgtttctcttttttgctggtcctcagggccctgctggcccccagggagcccctggacccgctggtgaagaaggcaagagaggtgcccgtggagagcctggtggcgttgggcccatcggtccccctggagaaagagttaagtgaatgtggaggctccatcccatggggcctgtgacctcgagagggaagtggagtccttgtggtccgtgttctggtcaagtcccgtgacttttccgcatgtcatcctcctctttctccatcctctccgcgggagagggagtctgatcccgagttgtgccgccaaccaccagactgacatgaaatagtctgagctccttcccaggaagcggggcaggctccagaagttaacctctgagaatcctgcaggccacagctgctccccagaaattggggttggtgggttagtgggatggacccactggagcctggctgggttgggctgttctcactcactgcctctcctccctgtggctccttagggtgctcccggaaaccgcggtttcccaggtcaagatggtctggcaggtcccaaggtgagtgggagaagaggggctggggtcctccctgcatcgctgaggtcacatggtatcccactgactccctgtgtacccttgtagggagcccctggagagcgagggcccagtggtcttgctggccccaagggagccaacggtgaccctggccgtcctggagaacctggccttcctggagcccgggtaagtagcagagctgctgttgcccttggcttcagaccctcaggcccttcctggctggctccttccagccctgcactgccaggattgggaggtcctggggccggctcctgaccccaccctcttctctctcctgaacaaagggtctcactggccgccctggtgatgctggtcctcaaggcaaagttggcccttctgtaagtctatcctctgagggctgctaggagggtggggggatctccctggggaagcaagggaaaagagagatggagtttgggttagggaggcctgaagtactgtgaattttgagaattgtgacgagggggtagatggtaggcactggggccagatgtaacctgtgcagtagctgtgagcactgaaaatgccaccccagtatgcattcggggcttatccttgggggaatgatgacatcgtgtgtgcactttctggggcagctttctaagctcagcggtgtcttgttgtagatgggcccatgggtgtgatgtggtcaatcctagatgctgagcatgtgtggctggtgccatgtcctggcctgccatgtaggcccttagtggatgttgggtggatggatgtggtcagagtgtctatgttctgagaatggtgttctgtctttcagggagcccctggtgaagatggtcgtcctggacctccaggtcctcagggggctcgtgggcagcctggtgtcatgggtttccctggccccaaaggtgccaacgtaagtaataatttgctcttctatttccttccatgtggtgctacctacctccctgccctcttggggaaagggctgggtcctgagtagagtttacccagggacagtgatgagtggggctcctgtgccatgggtggcagtgggggtctgtatgtgatttggggaaaatccatggccccacagagcctcggggcattgcggccataattgttccatgtggcagtgccagcaggctggttgccattatggcccctgaacagaagagaagggctgatactttgctttatcttggctgtccatcaggatgtggccccaggctcagtccctgcagcccgctctgcccccaactccctcccaaaccatccgcctcatggccctgccctctctttccttcagggtgagcctggcaaagctggtgagaagggactgcctggtgctcctggtctgagggtaagtatccttccccgctgcccatgacttggtggtggccgggcatctgcagggaggacaggggaacggcctccccatggcatggtcccgggacccctcagtattgagtgttgatctctgtggctagaccccatgctggctgggcctttgggtgtctacacagggagacttctgtttgccattggtcagcaggccggggagctggggaaggcttccatgctgagaacagctaagaaaagacggggccctgggaaggaagggaggggaaggtgtggaaatggagctcagctggggtaccgtggaggtctggaaactctgggccagaagtacctttgcccaatcctagggggactgcaagcgggaagaaaagcgtgtcattggacttttctttttctcttctgtctagggtcttcctggcaaagatggtgagacaggtgctgcaggaccccctggccctgctgtaagtacctgcccagcctccccaggtggccctgggggcaggggctgggaggggtgggggtgggagagcccatccattaatggagctgacagatgtgaatgtgggctgagctgatacaccagactcactctgagctgaggcagggtgtcccaggaggctgtgtggacccacattggtggagaggagtgtgggtggctgatgggagtgcagggaggcatgcatgcactgtctgagtggtgcaggaagacgcctgtgctgcccaccctgctgacctcccctgggccctactagctgtggctctcagggtctctggaacactggctcagctcaccttttcttttccactgcagggacctgctggtgaacgaggcgagcagggtgctcctgggccatctgggttccaggtaggtggctggaccaggctctctgtgtcagtcctttgccatacccagggctccctggacagcagcaggcactatcggtggagggcccacacctcttgcagtgtccaggcatcgagccttccctgcacccctggctgtcactgctgctgcttcctttctttgggtctgccctatactgtgcctccctgggggccagggcagcaaactcactcctttgctaacgcttgtcacttcggcttctagggacttcctggccctcctggtcccccaggtgaaggtggaaaaccaggtgaccaggtgagtatggggctctttggacctgcaacctgtttagatgggaaggtcttttctgatgcctaggaggcaagggcaagagggcatgaggagcctgtgaggcctgggaatgtctggacccatgtcccagcctccacagatgacacaatcccatggaggagtgatattcagccctgctgtggagaattgttcaggggtctgtgatatgaagccttcactctcacacatctttctttgttctccagggtgttcccggtgaagctggagcccctggcctcgtgggtcccagggtgagtatcctgttggccaatgcgggctgcctccttgggcctgccctgggtcctatgctcctgctcctttccccacctccctgcttctccctggaccttctcccccactgctgttggttgatcacttcttggtgtctctgccgcagggtgaacgaggtttcccaggtgaacgtggctctcccggtgcccagggcctccagggtccccgtggcctccccggcactcctggcactgatggtcccaaagtaagtgaggctgcatccagtaggggtcttcgtggtagcctggagtcccactgagcaggagagaggagcgggctcaggaggaatgaagaacagaagtggggggagctggaaaggaggtctacatgggaggaagggaaggaagaggggtttggggcctggttacccaggctccatgaacatgggttcagggagaggtgctgtccactacagactccctcttacctccctccccagggtgcatctggcccagcaggcccccctggcgcacagggccctccaggtcttcagggaatgcctggcgagaggggagcagctggtatcgctgggcccaaaggcgacagggtaagtactgaggttacagcctcctcaccaaagctgtggctttgccaatgtcctgccccttgtgatcgcttccgttcccttatggcacctggtgatgaaggtttctgttagccctttttgaggagcttaaagactcctttccaaagctccctgccttttagtgacatcctttcccctgttccttcatctcacccctgctgctcctcacccaccctgagaccacagcaaattcctcttgggcagggactgggctttccctagcaccccagcctgggtgggactgagcaaaccatgggggtcctggggtgcctggctgaggcggctggttttctcttccctcagggtgacgttggtgagaaaggccctgagggagcccctggaaaggatggtggacgagtaagtgaatgcgggctgctggactgctgggcattaggatcctagccctgcacccaggagagcaggagagagggtctgggcagtctgccactggggtccctggtcctgtctctgtcggggctgggcaactgcagggacttctctgttaaaatggggccagagggtaagtgggagctctggaggcggtgggagcacgcaccaaggttggcttggtgccgggccgcacgtgctcggctggctcagcctgcctccctcacctctacctgctctccccgcagggcctgacaggtcccattggcccccctggcccagctggtgctaacggcgagaaggtgagtcccggctcctttcctctccacaccttgcctccctgtcacacctccttcttatctcctgccaaaggggttctgtcttctcctccctcaccactgtcaccctcggccaagggctaggagtgaagagggggccctctcagaagtgaagccgctggcagtgttccctgttgggtggggcaactgggctggggtaaacacacattcagcagaggccctcgagagggtgcgggtatgggctgcacagtaacacaggctgtgcagggggacctggagcccccttcccacgagcaaggccccccaaatgcactttgccctctcccactctgcctccccaccttcttaccccagctcttcctcccttccccaccctcagggagaagttggacctcctggtcctgcaggaagtgctggtgctcgtggcgctccggtgagtgtctgcccctctgagcctggctctgccgaggcccctgggaaccagagagccagggagtcagtgcaggccctcatgctgcctggtggccctgtgtgctgccaggcactcggtccctccctacccgctgggtctagggtgggaggagagatgggaagggaagggggaaggcacgtcactcccatcatgtgttcagggtgagggcttttgggttaacagagcctctgcctgcgttcaggactaagggctgctttcagatccccgtctctggggaacaggaggctgggcagggccacggggctcttggaggggagcagaagcaggtcaggcagcggggcctgactctcgccatgcccccttctctcacagggtgaacgtggagagactggcccccccggaccagcgggatttgctgggcctcctgtgagtatctctgtccatcctcctgggtacctccactcaggccagttccacatccagcaccctcgggcatcggagcttgtcagggagggaaatggatgctcctctcctctcctttcccgcctccatactaatagaaccatcatgtccagaccaggacacacacgcagatactcacagagtctcccgctcttctggaagagctccagggttttagcctgcccctcattcacctgcttcctccttccccattatagggtgctgatggccagcctggggccaagggtgagcaaggagaggccggccagaaaggcgatgctggtgcccctggtcctcagggcccctctggagcacctgggcctcaggtgggtaacgctgcactccaagaattgttccctcaaggaagggctcctggcgtgcagatgggaaggccccagcaggctgcgcagaggatggttcgcaggcctgggaacacccccatgttggtagaaggagcttccatgtggcatgtgggctgtgtgggtggggtgtagggactgacagagtacaggctggccacagccagccagaaccaagctgctgatctcctggggaggagggggcggtggcaggaagagcttcccgggaggccagaccccagaccggttctgtggttgcctgacaggcttctcctagaacacaagtctcctgtggcagaggggacagagctgcctgtggacgcctcttcaggctgggtttttagtgccaagaaagctgcatcttcgaaaacctcaggggtccattgttggggctcagacagaagcccaccgtcttccttgggccactgggcctcactgtctccctctttcctttccagggtcctactggagtgactggtcctaaaggagcccgaggtgcccaaggccccccggtgagtgaggcctctgacaccccaccctgcacctcacaaagaggcctggccccagaggctcccatggcggggggtgttctgggatgcccccgactgttttcccagccctgcgttggtccccagcctaagcccacccacagccaggtgggagagagggcgcctgtgggctgggtgcactgtggtcccggattccccagcccgaggcttgtccctcgttcagctacctgaagtgctgactgtggaaaccggagcagggaaacagcctgtgcctgcttctatgaccagacccgggggccctttctccctccctggatcccccttgcgggcttgggatccctcgccctttccataccaggctctgagaccacccccgccccccgcccattttaatctcaaacctcctgagggcttgaggttctcaggggctctcctctccccacacagggagccactggattccctggagctgctggccgcgttggacccccaggctccaatgtaatggatgctcctggcatgagaggcacaggcaggcatggaatagcctggcccagcacccaacaggtctggagagctccaggctggccttcactcctctgagtccgttcctgccccctggaggagtagctaactgatcatggggacctatcccctgagggtggctgggggcagggttgtggccctgctgtcagcaatgggtggctgccctgctggcttggtgggcaggacagggctgccatgattaccttcatcctggacagatgtttcttgagcatgttctgtgcacttgtctgtgtgctttaaagcaagtccgcatgtcctgggtcgctctcagggagtttgcatggagggagcagtggtgatgaactcactgggttcaccagcgccataggcagagggagcgagtgctgggggcaagctcaggggtcagaggggctgggctggagcggctcctgaggaagggggattgaggacaaagggaggagggaggagagattccaggtagatgggagaaagcaagcaggagctggagaagttggggctgtctgcccagagctgtctcacatggtgagaaggttgcagcggtagattggggttgggggggttaagctgcctgcccttagtgtggggaggtagagaagcccccagagaggaaactgctgtcactgaggccacagtgactttggcagcctggatgaggaagggtgagatgagtcctcacttcccgcattttctccttctagggcaaccctggaccccctggtccccctggtccttctggaaaagatggtcccaaaggtgctcgaggagacagcggcccccctggccgagctggtgaacccggcctccaaggtcctgctggaccccctggcgagaagggagagcctggagatgacggtccctctgtaagtccctcaccaggcccatgccaaggtccctgggagcagggtcgtgagtggcttctgagctcacagagcatggggtaggagggagcaggggccgtggggatgccagggagcggggctgcacagacagagctgtgctgagaggacgaagaggctgggccactgtcagttctcatctcctgcctctcctctctcagggtgccgaaggtccaccaggtccccagggtctggctggtcagagaggcatcgtcggtctgcctgggcaacgtggtgagagaggattccctggcttgcctggcccatcggtgagtgtggggtatccctccctccatccaagctggccctgcctgccaaggcttctacctccctcagcaccctcaggactgtcccttgtctgccctctcctgaagggtcagtgggccctgggcaggggtgcttaccacttgcactcatcatccttgtctctgtcctccagggtgagcccggcaagcagggtgctcctggagcatctggagacagaggtcctcctggccccgtgggtcctcctggcctgacgggtcctgcaggtgaacccggacgagaggtgagcagtgagaccccctggggtggccctgattggggagaggggccctgtgagtctctgtgctgggtcagcaaggacaagccccagtcagggcctcggagaagggggcggcagcgctggccgacaggcgaaagcctaggtacaatgggaaggttgtcggggagagagacgggcatagagaccaagggctgcttctggaaggaggagggaaacttggtgaggaaactttggcttcaaagtgtgagtgagttgggcagaagaggagaggcctgggcttctgagaggggctgggggagcagagggggaggtgggcaggaagcagctctaagtgcattcttgtttcactttgtccagggaagccccggtgctgatggcccccctggcagagatggcgctgctggagtcaaggtgagtgtctggtgtctgtgtgtgcagtgggttggggaggacattgcctcgggcctgacaggtcagctgggggtggcaggttggaacaagtctcatctcagcctagaaggaccttctgttcctgtctcttctggaacattcttctctgagcctgagacctctctcctgacagggtgatcgtggtgagactggtgctgtgggagctcctggagcccctgggccccctggctcccctggccccgctggtccaactggcaagcaaggagacagaggagaagctgtaagtatcctggaattcagtaaaagccgccttcccctgcgcggtggggctgaggcagtccctgggtttccgcagtctctggactaaggagcagtggcctcagatgcagaggaggcccccacctgtcctggcttttctctgacgctgcgctcactctctcctcagggtgcacaaggccccatgggaccctcaggaccagctggagcccggggaatccaggtgagtatccaagtgtcctgcactgagtccccaccagggataggctgggagggcagccagcctccaggtggttcctggcctccagccctgtgtttccggggattcctcagcttgggtgggacaggagggggctcctgtcctggccctgacctgactcaatcggtgtctgtcttgttcccagggtcctcaaggccccagaggtgacaaaggagaggctggagagcctggcgagagaggcctgaagggacaccgtggcttcactggtctgcagggtctgcccggccctcctgtgagtgtcactgcctgcgtgggacttcccgaggcctcctgccacacagagcccacttgagctccctgtgctgccaggacagcttgggatcaccctaagcagtttctaggatttcctcagggctggagggaggaggaagtggaaagggaatggggctgggacataaagctgttcccccagctcccagaatatagatagatatgtctgtgctgaccgtggccttttgcctcttccttctacacagggtccttctggagaccaaggtgcttctggtcctgctggtccttctggccctagagtaagtgacatggagttggaagatggagggggcccttcagagagtgtgggcctgtgttcccatggggagggaaatgctgctgcttctggggaagctgtgggctcaggggtcctcactcagtaatgggggcaggactggctcatgtgcctatggccagaaaagcgcctgaggccacaatggctgtaagacaaacatgaatcagcctctcgctgtcagacagaacagcattttacaaagaggagcttaggagggtaggcaagccatggagctatcctgctggttcttggccaaatagagaccaacttagggttccatgactgagcatgtgaagaactgggggcggagtggctggtgctatcaggacagccacctacccagccccagcgactccccagccttccctgtggtgaccactctttcctcacgacctctctctcttgcagggtcctcctggccccgtcggtccctctggcaaagatggtgctaatggaatccctggccccattgggcctcctggtccccgtggacgatcaggcgaaaccggtcctgctgtaagtgtcctgactccttccctgctgtcgaggtgtccctaccatccgggaggcttgagctcttttttgctcagggcctcttttagggcatcagcctgcagctaacagtgatggcatcctttatcctgaggtctcctcagaggtcacagggcccatgatcagtgctgggaaactgaagagaagggctaaggaagaaatagacatggtgctgtggtttccttggtcctcgcctgctacacctccgccccacccatggggctgggaagagggacactctagtacattctagcaaatggggatggacatggaggggcactttcacacaatcctggctgatctctctgtttcctgctgcagggtcctcctggaaatcctgggccccctggtcctccaggtccccctggccctggcatcgacatgtccgcctttgctggcttaggcccgagagagaagggccccgaccccctgcagtacatgcgggccgaccaggcagccggtggcctgagacagcatgacgccgaggtggatgccacactcaagtccctcaacaaccagattgagagcatccgcagccccgagggctcccgcaagaaccctgctcgcacctgcagagacctgaaactctgccaccctgagtggaagagtggtaagcttggagaacaggatcccctgccccgggaagcagggagtcatcccttaggcctagcagcaagggaggagatgccccctagtacagggcagagctgggcctggaagtttccgccagagggttcctctcttatttcacagcagagaagctgcagccctggcccctgtcctgccatggctacctggccgaggtgacctcagggtggactccatccaccagctgggcactgcttctgctctctttgcatgtgttcttccttagggctggacttagctcatgcagatctccctgcccctgcatcctcccaggtccccctcctttcaggccacatgtgaacctcatcccttgtccctgtaggcctctctgtctctttcagtcaggcctgggtctctcaagcttttgtgtctgtgcctgtctgagcccccatgggtgctgcctcttccccctgcaggagactactggattgaccccaaccaaggctgcaccttggacgccatgaaggttttctgcaacatggagactggcgagacttgcgtctaccccaatccagcaaacgttcccaagaagaactggtggagcagcaagagcaaggagaagaaacacatctggtttggagaaaccatcaatggtggcttccatgtgagtacctgggtgccctagatgatgagcagagatggctcctcaaactctttcttttctttctccctggaagcttttagcaccttccccatattttcctccagttttctgttgggcttgagaggagggaaagaggaggaaaagtattttttccccacgtggaggtgggaaaagaggtcctctgagcttgctccactcctggaagcaaaaatgtccaactagctccctgctgccccagtacccttgaggtccttgaaccatgaactcttggcagcccctacagcccctggtcccattgaatgccagctcccaggcctcacactgccgctctctgccccaacagttcagctatggagatgacaatctggctcccaacactgccaacgtccagatgaccttcctacgcctgctgtccacggaaggctcccagaacatcacctaccactgcaagaacagcattgcctatctggacgaagcagctggcaacctcaagaaggccctgctcatccagggctccaatgacgtggagatccgggcagagggcaatagcaggttcacgtacactgccctgaaggatggctgcacggtgagtggggctgccagagagaagagctgcctgtgcccaaactgcctggagcagggctgagggttggcccgcggcagctgtcaggtcctaaagtgacaggatcatcagaggcatgagtttgagggtcatgtagagaagataggctgagtgacaggtgagagagaggcacatatcattccatcttctccattcccctggctcaggggaacaaaaccctacctggaacccagtgactactgtagaagtgttctcgcaatgtgtacagggtgaagaagcggtcacaggttgggagctcactgtggggagtggggaaggaggggaagggcagggtggagaagggccctgccgctaaggataggagttgaagtggagaggcctttggcaagccaagaagaggtctcaggagccccctcagtgtggttcaaccttgtgggctctgatgctcgccagtttgttcagttttgggcttctgggcagctggaactgggtagcaaggcatctactgaacagagcctcctccttttttctcccctagaaacataccggtaagtggggcaagactgttatcgagtaccggtcacagaagacctcacgcctccccatcattgacattgcacccatggacataggagggcccgagcaggaattcggtgtggacatagggccggtctgcttcttgtaa

SEQ ID NO: 13 - DNA, 26 bp, Artificial
  primer for Rattus rattus
ggtacgaattcatgattcgcctcggg

SEQ ID NO: 14 - DNA, 25 bp, Artificial
  primer for Rattus rattus.
cagcactgtccattggtccttgcat

SEQ ID NO: 15 - DNA, 25 bp, Artificial
  primer for Rattus rattus.
aggaccaatggacagtgctgctctg

SEQ ID NO: 16 - DNA, 29 bp, Artificial
  primer for Rattus rattus.
ggtacgaattcatgattcgcctcggggct

SEQ ID NO: 17 - DNA, 24 bp, Artificial
  primer for Rattus rattus.
taaggatccaactttgctgcccag

SEQ ID NO: 18 - DNA, 24 bp, Artificial
  primer for Rattus rattus.
aatggatccaactttgctgcccag

SEQ ID NO: 19 - DNA, 24 bp, Artificial
  primer for Rattus rattus.
gtaccgaattctcagaactcacag

(19) United States
(12) Patent Application Publication
(10) Pub. No.: US 2008/0166798 A1
(43) Pub. Date: Jul. 10, 2008

(54) Trimerizing Polypeptides and Their Uses

(75) Inventors: Audrey McAlinden, St. Louis, MO (US); Erika Crouch, St. Louis, MO (US); Linda Sandell, St. Louis, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Barnes-Jewish Hospital, St. Louis, MO (US)

(21) Appl. No.: 10/569,246

(22) PCT Filed: Aug. 21, 2004
(86) PCT No.: PCT/US2004/027381

§ 371 Date: Mar. 18, 2008

Related U.S. Application Data
(60) Provisional application No. 60/497,054, filed on Aug. 22, 2003.

(51) Int. Cl. C12N 5/00; C07K 14/00; C07H 21/04

(52) U.S. Cl. 435/325; 530/402; 536/22.1

(57) ABSTRACT

A method for trimerizing collagenous molecule monomers comprising the step of contacting a collagen domain and a non-collagenous trimerization domain is provided. In addition, methods of trimerizing heterologous peptides is provided. Trimerizing polypeptides, vectors, cells, and trimerized polypeptides are also provided.

### CROSS-REFERENCES TO RELATED APPLICATIONS

This application claims priority from Provisional Application Ser. No. 60/497,054 filed on Aug. 22, 2003, which is incorporated herein by reference in its entirety.

### STATEMENT REGARDING FEDERALLY SPONSORED RESEARCH OR DEVELOPMENT

This invention was made in part with Government support under NIH Grants AR-36994, HL-29594, and HL-44015. The Government has certain rights in the invention.

### INCORPORATION-BY-REFERENCE OF MATERIAL SUBMITTED ON A COMPACT DISC

The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The subject matter of the Sequence Listing is incorporated herein by reference in its entirety.

### BACKGROUND OF THE INVENTION

1. Field of the Invention

The invention relates to polypeptides capable of forming trimers. Methods of using such polypeptides are also disclosed.

2. Description of the Related Art

The type IIA amino (NH₂)-propeptide is encoded by eight exons. The translated protein consists of a short globular domain, a 69 amino acid von Willebrand factor type C (VWfC) cysteine-rich domain, a minor collagen triple-helical domain containing 26 Gly-X-Y repeats and a short telopeptide domain which links the minor collagen domain to the major collagen triple-helix. Trimerization of most fibrillar collagens is dependent on the globular carboxy (COOH) propeptide for the recognition and association of the three polypeptide chains resulting in registered nucleation of triple-helix formation in a zipper-like fashion from the C- to N-terminus. Functions proposed for procollagen NH₂-propeptides include the regulation of collagen fibrillogenesis and a feedback control of net collagen biosynthesis. It has also been proposed that the NH₂-propeptide of type IIA procollagen regulates growth factor activity in the extracellular matrix.

Trimeric assembly of fibrillar NH₂-propeptides affects protein valency and stability, which are important for function in vivo. This emphasizes the importance of a procollagen COOH-propeptide, or indeed other protein domains with similar function, to drive this trimerization process.

Pulmonary surfactant protein D (SP-D) is predominantly assembled as dodecamers, consisting of four trimeric subunits cross-linked by disulfide bonds. Each SP-D subunit contains an amino-terminal cross-linking domain, an uninterrupted triple-helical collagen domain consisting of 59 Gly-X-Y repeats, a trimeric coiled-coil neck domain and a C-type lectin carbohydrate recognition domain (CRD). Trimerization of SP-D subunits and subsequent oligomerization of these trimeric subunits to form higher order multimers, results in increased valency of the CRD, an essential pre-requisite for high affinity ligand binding. The neck domain of SP-D is the unit responsible for driving the trimerization of the three polypeptide chains of SP-D. It was demonstrated that a 35 amino acid sequence containing the human neck sequence was sufficient to form stable, non-covalent, trimeric complexes in vitro. The same sequence was found to be important for the association of the three CRDs of human SP-D; CRDs synthesized in prokaryotic cells without this neck domain were assembled as monomers.

The sequence of coiled-coil domains is characterized by a seven-residue repeat (commonly denoted (abcdefg)n) where positions a and d are primarily occupied by hydrophobic residues, positions e and g by charged residues, positions b, c and f by polar or charged residues, and n is an integer beginning with the numeral 1. The following Table 1 describes the hydrophobicity, polarity and charge of common amino acids:

TABLE 1

| Amino Acid | 3-letter code | 1-letter code | Properties |
|---|---|---|---|
| Alanine | Ala | A | Aliphatic, hydrophobic, neutral |
| Arginine | Arg | R | polar, hydrophilic, charged (+) |
| Asparagine | Asn | N | polar, hydrophilic, neutral |
| Aspartate | Asp | D | polar, hydrophilic, charged (−) |
| Cysteine | Cys | C | polar, hydrophobic, neutral |
| Glutamine | Gln | Q | polar, hydrophilic, neutral |
| Glutamate | Glu | E | polar, hydrophilic, charged (−) |
| Glycine | Gly | G | aliphatic, neutral |
| Histidine | His | H | aromatic, polar, hydrophilic, charged (+) |
| Isoleucine | Ile | I | aliphatic, hydrophobic, neutral |
| Leucine | Leu | L | aliphatic, hydrophobic, neutral |
| Lysine | Lys | K | polar, hydrophilic, charged (+) |
| Methionine | Met | M | hydrophobic, neutral |
| Phenylalanine | Phe | F | aromatic, hydrophobic, neutral |
| Proline | Pro | P | hydrophobic, neutral |
| Serine | Ser | S | polar, hydrophilic, neutral |
| Threonine | Thr | T | polar, hydrophilic, neutral |
| Tryptophan | Trp | W | aromatic, hydrophobic, neutral |
| Tyrosine | Tyr | Y | aromatic, polar, hydrophobic |
| Valine | Val | V | aliphatic, hydrophobic, neutral |

The crystal structure of the neck and lectin domain of human SP-D has been solved and the coiled-coil region was visualized as a stretch of greater than 28 amino acids (Arg²⁰⁸-Pro²³⁵) consisting of approximately 8 helical turns.

Earlier work suggested that the presence of valine at the d positions favors the trimeric assembly of human SP-D. It was further suggested that the unusual fourth heptad, which contains Phe²²⁵ and Tyr²²⁸ in the a and d positions, respectively, might serve to initiate trimerization. However, no valine residues are found in the neck of rat SP-D. In addition, it was observed that deletion of the conserved fourth heptad repeat does not prevent trimerization of recombinant rat SP-D secreted by mammalian cells. On the other hand, internal deletions of residues 207-214 or 214-221 within the neck domain were found to block trimerization and indicated that sequences amino-terminal to Phe²²⁵ were required for trimerization.

The requirements for collagen trimerization and folding vary with the collagen type. Generally,.fibrillar collagens and type IV collagen require the presence of globular sequences C-terminal to the triple-helical domain to initiate chain registration. However, trimerization of type XII collagen is dependent on specific post-translational modifications of the collagen domain while chain association of the membrane-associated collagen, type XIII, occurs in the N-terminal region. Re-folding experiments on collagen type III indicated that inter-chain disulfide bridges at the C-terminus of the triple helix was sufficient to function as a nucleus for the re-folding of the triple helix. These findings suggest that the sequences requires for driving collagen trimerization can be manipulated as also exemplified by our ability to trimerize a procollagen amino propeptide using the a-helical coiled-coil domain of rat SP-D.

Two studies describe heterologous trimerization of collagen sequences to drive the trimerization of collagen sequences. Frank et al. (J. Mol. Biol. 308:1081-1089 (2001)) utilized the bacteriophage T4 fibritin foldon domain to synthesize a chimeric protein consisting of a synthetic collagen peptide (ProProGly)₁₀ fused to the N-terminus of the foldon. The foldon domain, which consists of 27 amino acids and forms a β-propellar-like structure with a hydrophobic interior, was sufficient to drive the trimerization and correct folding of the synthetic collagen domain. Another study (Bulleid et al., EMBO J. 16:6694-6701 (1997)) showed that the COOH-propeptide of type III procollagen could be replaced with a transmembrane domain without affecting the folding of the collagen triple helix.

In addition, U.S. Pat. No. 6,190,886 to Hoppe et al. describes polypeptides comprising a collectin neck region, or variant or derivative thereof or amino acid sequence having the same or a similar amino acid pattern and/or hydrophobicity profile, are able to trimerize. Such polypeptides may comprise additional amino acids which may include heterologous amino acids, for example, forming a protein domain or derived from an immunoglobulin or comprising an amino acid which may be derivatized for attachment of a non-peptide moiety such as oligosaccharide, and may form homotrimers or heterotrimers. Heterotrimerization may be promoted by gentle heating, e.g. to about 50° C., then cooling to room temperature. One use for the polypeptides is in seeding collagen formation. Nucleic acid encoding the polypeptides and methods of their production are provided.

However, the trimerizing polypeptides described above are limited in their use because they are difficult to use to trimerize polypeptides with similar effect in vivo as well as in vitro. Because of this limitation, uses of the above trimerizing polypeptides in vitro do not accurately translate or cannot be used for therapeutic or other actions in vivo. In addition, the above described trimerizing polypeptides may not support normal folding of a procollagen propeptide domain, such a domain greatly enhancing the normal folding (folding found in vivo) of collagenous proteins both in vivo and in vitro. Additionally, many of the above described trimerizing polypeptides comprise a functional SP-D lectin domain which negatively affects the function of trimeric polypeptides in vivo.

Thus, what is needed is a minimum sequence of a trimerizing polypeptide capable of trimerizing procollagen propeptides to form collagenous molecules, and capable of trimerizing other oligomers, enabling use of such trimerizing polypeptides both in vitro and in vivo.

### BRIEF SUMMARY OF THE INVENTION

Accordingly, it is an object of the invention to overcome these and other problems associated with the related art. These and other objects, features and technical advantages are achieved by providing a minimum sequence for trimerizing procollagen propeptides and oligomers which take on the same comformation in vitro as in vivo.

This invention provides a method for trimerizing collagenous molecule monomers comprising the step of contacting a collagen domain and a non-collagenous trimerization domain. Preferably, the non-collagenous trimerization domain comprises a 14 amino acid sequence corresponding to the first two heptad repeats of the neck domain of mammalian pulmonary surfactant protein D. More preferably, the mammalian pulmonary surfactant protein D is rat pulmonary surfactant protein D. Altematively, the mammalian pulmonary surfactant protein D is human pulmonary surfactant protein D. Most preferably, the 14 amino add sequence is SEQ ID NO: 1.

In accordance with a further aspect of the invention, a method for trimerizing collagenous molecule monomers without a dimeric intermediate is provided comprising the step of contacting a collagen domain and a non-collagenous trimerization domain. Also provided is a method for producing a native conformation of NH₂-propeptide of type IIA procollagen in vitro comprising the step of contacting a collagen domain and a non-collagenous trimerization domain.

In accordance with yet another aspect of the invention, a trimerized collagenous molecule monomers produced by contacting a collagen domain and a non-collagenous trimerization domain is provided. Additionally, a NH₂-propeptide of type IIA procollagen produced by contacting a collagen domain and a non-collagenous trimerization domain is provided.

In accordance with yet another aspect of the invention, a polypeptide having the sequence of SEQ ID NO: 1 is provided. Further, a trimer comprising three collagenous molecule monomers is provided, said monomers consisting of a truncated SP-D domain of SEQ ID NO: 1. In one embodiment, a collagenous molecule monomer consisting of two heptad repeats of SP-D is provided, the heptad repeat having the formula:

(abcdefg)ₙ

wherein positions a and d are occupied by hydrophobic residues; positions e and g by charged residues; positions b, c and f by polar or charged residues; and n is 2. In another embodiment, a collagenous molecule monomer comprising two contiguous sites for BS³ cross-linking within the fourth heptad repeat of SP-D is provided, the heptad repeat having the formula:

(abcdefg)ₙ

wherein positions a and d are occupied by hydrophobic residues; positions e and g by charged residues; positions b, c and f by polar or charged residues; and n is 4. In yet another embodiment, a truncated fusion protein consisting of two heptad repeats of SP-D is provided, the heptad repeat having the formula:

(abcdefg)ₙ

wherein positions a and d are occupied by hydrophobic residues; positions e and g by charged residues; positions b, c and f by polar or charged residues; and n is 2. In yet another embodiment, a truncated fusion protein consisting of three heptad repeats of SP-D is provided, the heptad repeat having the formula:

(abcdefg)ₙ

wherein positions a and d are occupied by hydrophobic residues; positions e and g by charged residues; positions b, c and f by polar or charged residues; and n is 3.

A further aspect of the invention provides a chimeric gene construct comprising a cDNA encoding exons 1 through 8 of type IIA NH₂-propeptide operably linked to a cDNA encoding the neck domain and lectin domain of SP-D. Additionally, a chimeric gene construct comprising a cDNA encoding exons 1 through 8 of type IIA NH₂-propeptide operably linked to a cDNA encoding the neck domain of SP-D. Still further, a fusion protein comprising a IIA NH₂-propeptide collagen domain and a 14 amino acid sequence of the SP-D coiled-coil neck domain of SEQ ID NO: 1.

In another aspect of the invention, a cell transfected with a chimeric gene construct is provided comprising a cDNA encoding exons 1 through 8 of type IIA NH₂-propeptide operably linked to a cDNA encoding the neck domain and lectin domain of SP-D. In another embodiment, a cell transfected with a chimeric gene construct is provided comprising a cDNA encoding exons 1 through 8 of type IIA NH₂-propeptide operably linked to a cDNA encoding the neck domain of SP-D. In addition, a stably transfected cell line is provided comprising a chimeric gene construct comprising a cDNA encoding exons 1 through 8 of type IIA NH₂-propeptide operably linked to a cDNA encoding the neck domain and lectin domain of SP-D. In yet another embodiment, a stably transfected cell line is provided comprising a chimeric gene construct comprising a cDNA encoding exons 1 through 8 of type IIA NH₂-propeptide operably linked to a cDNA encoding SEQ ID NO: 1.

In another aspect of the invention, a polypeptide is provided wherein the first amino acid sequence is SEQ ID NO: 1. Further, a nucleic acid comprising a sequence of nucleotides encoding a polypeptide according to the above. Still further, a nucleic acid is provided wherein said nucleic acid further comprises a vector. In another aspect, a host cell containing a nucleic acid encoding a polypeptide having SEQ ID NO: 1 is provided. Preferably, a nucleic acid is provided, wherein the encoding sequence is operably linked to a regulatory sequence for expression of the polypeptide.

In yet another aspect of the invention, a host cell is provided containing the nucleic acid encoding a polypeptide having SEQ ID NO: 1. In a further aspect, a trimer is provided comprising the polypeptide having SEQ ID NO: 1. In one alternative, the trimer is a homotrimer. In another alternative, the trimer is a heterotrimer.

In another aspect of the invention, a protein expression method is provided comprising expressing a polypeptide having SEQ ID NO: 1 from a nucleic acid encoding the polypeptide. In yet another aspect of the invention, a polypeptide trimerizing method is provided comprising forming a trimer comprising a polypeptide having SEQ ID NO: 1 following its expression. In one altemative, the trimer is a homotrimer. In another altemative, the trimer is a heterotrimer.

These and other features, aspects and advantages of the present invention will become better understood with reference to the following description, examples and appended claims.

### BRIEF DESCRIPTION OF THE SEVERAL VIEWS OF THE DRAWINGS

FIG. 1: Amino acid sequence of human type IIA procollagen NH₂-propeptde. Sequence begins at the signal peptide cleavage site, numbered as the first amino acid (Q). Arrows indicate exon (E) boundaries. The cysteine-rich, vWfC domain encoded by exon 2, is shown in italics. Underlined amino acids in the region encoded by exons 3-7 denotes the minor collagen domain containing 26 Gly-X-Y repeats and a 4 amino acid interruption between exons 4 and 5. The telopeptide domain connects the propeptide to the major procollagen triple-helical domain and is encoded by exon 8.

FIG. 2: Production of IIA/SP-D chimeric construct and predicted structure of the recombinant fusion protein. cDNA encoding the collagenous domain of SP-D was replaced with cDNA encoding the type IIA NH₂-propeptide, the eight exons of which are represented by numbers. N=trimerizing coiled-coil neck domain; CRD=carbohydrate recognition domain. Grey shaded area indicates the cysteine-rich domain encoded by exon 2.

FIG. 3: Purification of IIA/SP-D fusion protein. IIA/SP-D produced by stably-transfected CHO cells was purified from 1 liter of conditioned media by maltosyl-agarose chromatography. (A) silver stain showing the presence of IIA/SP-D in EDTA-eluted fractions 4-7 after SDS-polyacrylamide gel electrophoresis under non-reducing conditions. Monomer (M) size of the protein is approximately 45 kD compared to globular protein standards. Stable trimers (T) of IIA/SP-D were also detected. (B) Anti-Exon 3-8 immunoblot of an EDTA-eluted fraction. IIA/SP-D immunopositive bands were detected under reducing and non-reducing conditions (+/−DTT). Slower migration after reduction is due to disruption of the intra-chain disulfide bonds in the lectin domain of SP-D.

FIG. 4: Collagenase digestion of SP-D and IIA/SP-D. (A) Coomassie blue stained gel showing SP-D and IIA/SP-D+/−bacterial collagenase. Lower molecular weight, collagenase-resistant bands are denoted by bands 1, 2 and 3. * indicates the collagenase enzyme. (B) Schematic showing the location and amino acid sequence of the major collagenase-resistant bands in SP-D (band 1) and IIA/SP-D (bands 2 and 3).

FIG. 5: Purification of the IIA-NH₂-propeptide by MMP-9 or enterokinase digestion and maltosyl-agarose chromatography. (A) Schematic showing the location of MMP-9 (gelatinase B)-specific cleavage sites within the telopeptide region of the IIA NH₂-propeptide and the position of the engineered enterokinase (EK) cleavage site within the telopeptide of the mutant IIA/EK/SP-D protein. Numbers represent the exons of the IIA NH₂-propeptide. (B) Silver stained gels showing the presence of IIA NH₂-propeptide in the column flow-through (FT) after MMP-9 or EK digestion. Neck and carbohydrate recognition domain (N+CRD) fragments were present in the EDTA eluate (E).

FIG. 6: Circular dichroism spectroscopy of the IIA NH₂-propeptide collagen domain. IIA NH₂-propepfide purified from enterokinase cleavage of IIA/EK/SP-D fusion protein was analyzed by circular dichroism (CD) spectroscopy. (A) CD spectrum shows a large positive ellipticity at 225 nm, indicative of a collagen triple helix. (B) Melting temperature of the collagen helix in the IIA propeptide is approximately 42° C. as shown by the decrease in ellipticity with increasing temperature from 5° C. to 70° C. θ=mean residue ellipticity.

FIG. 7: Covalent cross-linking of IIA/SP-D or IIA NH₂-propeptides synthesized with or without the trimerization domain of SP-D. The transition from monomers (M) to trimers (T) through a dimer (D) intermediate with increasing concentrations of BS³ cross-linker is shown for IIA/SP-D. The same pattern is shown for the purified IIA NH₂-propeptide that was synthesized attached to the neck and lectin domain of SP-D and then subsequently purified by MMP-9 treatment. *=MMP-9-derived product not immunoreactive with either IIA or SP-D antisera. The IIA Western blot shows that type IIA NH₂-propeptide produced in transiently-transfected CHO cells without the trimerization cassette of SP-D exists only as monomers in solution.

FIG. 8: Amino acid sequence of SP-D a-helical coiled-coil neck domains from different species and schematics showing mutant IIA/SP-D fusion proteins containing a premature stop codon within the coiled-coil domain. Amino acid sequence of the coiled-coil neck domain shows the presence of four contiguous heptad repeats. Positions a and d, generally represented by hydrophobic residues, are indicated. Schematic below shows the complete sequence of rat SP-D neck domain attached to IIA NH₂-propeptide at its N-terminal side. The coiled-coil sequence ends at the last proline residue (Pro²³⁵) and proceeds to the sequence encoding the carbohydrate recognition domain (CRD). Underlined amino acids represent locations where the codon was replaced by a premature stop site in the cDNA sequence. Each mutant (m) protein consists of the full-length IIA NH₂-propeptide sequence fused to either one (mIIA-211), two (mIIA-218) or three heptad repeats (mIIA-225) of the coiled-coil neck domain. IIA NH₂-propeptide devoid of the neck sequence (mIIA-203) or attached to the “full-length” sequence previously reported to drive trimerization (mIIA-237) were included as controls. Amino acids labelled with a stars (*) indicate residues that may participate in electrostatic interactions to stabilize the coiled-coil at its N-terminal end: Arg²⁰⁸ to Glu²¹² (i to i+4 intra-chain) and/or Asp²⁰³ to Arg²⁰⁸ (i to i+5; g-e′ inter-chain).

FIG. 9: Chemical cross-linking of IIA NH₂-propeptides fused to different regions of the SP-D coiled-coil neck domain. To determine the minimum sequence of the coiled-coil neck domain that can function as a trimerizabon domain, increasing amounts of cross-linker (BS³) were added to each mutant protein. Western blotting and immunolocalizabon using the anti-IIA polyclonal antibody was used to detect the protein. IIA NH₂-propeptides devoid of the coiled-coil neck domain (mIIA-203) or containing one heptad repeat of the neck domain (mIIA-211) were shown to exist only as monomers (M) in solution. However, for the IIA NH₂-propeptides attached to either two (mIIA-218) or three (mIIA-225) heptad repeats, trimer (T) formration is noted through a dimer (D) intermediate with increasing amounts of BS³. The mutant protein consisting of the IIA propeptide attached to “full-length” neck sequence (mIIA-237) was more efficiently trimerized at lower concentrations of cross-linker than that used for the other truncated proteins and, in addition, no dimer intermediate was detected.

FIG. 10: Production and chemical cross-linking of a collagen deletion mutant protein. (A) Schematic showing the collagen deletion protein (mIIA-coll-218) consisting of exons 1, 2 and 8 of the IIA NH₂-propeptide fused to the short, 14 amino acid sequence of the SP-D coiled-coil neck domain (represented by the diagonal-shaded box). (B) IIA immunoblot showing the presence of the collagen deletion protein from conditioned media of transiently-transfected CHO cells. There was no detection of dimers or trimers after addition of the highest concentration of cross-linker (BS³, 2 mM). Without the collagen domain (encoded by exons 3-7), the truncated fusion protein exists as monomers in solution.

### DETAILED DESCRIPTION OF THE INVENTION

### Application of a 14 Amino Acid Polypeptide to Trimerization

The present invention is a short, amphipathic helical trimerizing polypeptide VASLRQQVEALQGQ (SEQ ID NO: 1) derived from the rat SP-D neck domain which can drive the trimerization of a fibrillar collagen NH₂-propeptide as well as other propeptides and oligomers.

The present invention describes an efficient system for producing high levels of a correctly-folded NH₂-propeptide of type IIA procollagen. This approach could likely be applied to the synthesis other procollagen NH₂-propeptides, and other oligopeptides, which are difficult to isolate from tissues. Given that the propeptide is trimeric and correctly-folded, it will be possible to examine the contributions of valency to the biological function of this peptide. The ability to express a secreted trimeric propeptide without inclusion of the functional lectin domain of SP-D will also enable us to investigate the effects of the propeptide in in vivo models of tissue development and repair.

Such a trimerizing polypeptide results in a IIA NH₂-propeptide which is folded in vitro the same as it is in viyo. The amino acid sequence consists of the first two heptad repeats of the neck domain, which is in agreement with our previous deletional mutagenesis studies showing that amino-terminal regions of the neck domain are important for initiating trimerization (Zhang et al., J. Biol. Chem. 276:19862-19870 (2001)). This is by far the shortest sequence found to permit trimerization of a collagenous molecule, and the first to demonstrate the use of a heterologous trimerization cassette to support the normal folding of a procollagen propeptide domain.

High levels of a correctly-folded IIA NH₂-propeptide were produced using this system, which will enable the study its biological function in vitro. Establishing a minimum sequence of the SP-D neck domain that can drive tnimerization without inclusion of the functional SP-D lectin domain allows the study the function of the trimeric IIA propeptide in vivo. Knowledge gained from these findings may be applied to produce other procollagen propeptides or indeed other collagenous proteins for functional studies.

The polypeptide of the present invention is a 14 amino acid sequence derived from the first two heptad repeats of the α-helical coiled-coil domain of rat SP-D (SEQ ID NO:1). This polypeptide can drive the trimerization of a heterologous procollagen NH₂-propeptide sequence. Although IIA propeptides alone are secreted as monomers, a IIA/SP-D chimera with a truncated SP-D neck domain terminating at residue 218 was sufficient to drive trimerization. Truncations at residue 211 or 203, containing one or no heptad repeats, respectively, were secreted as monomers. This is the shortest sequence ever described to support the trimerization of a collagen sequence.

In addition, trimerization is accompanied by folding of the collagen triple helical domain and that, following cleavage from the SP-D sequence, the IIA NH₂-propeptide retains its trimeric conformation. Amino acid analysis revealed that approximately 80% of the potential proline residues in the Y position of the collagen sequence are hydroxylated, consistent with the formation of a stable triple helix. These levels of hydroxylation are comparable to that reported for the al chain of the NH₂-propeptide of type I procollagen extracted from developing bone (Fisher et al., J. Biol. Chem. 262:13457-13463 (1987)). In addition, the melting temperature of the collagen helix within the recombinant propeptide was similar to other comparably hydroxylated collagens, approximately 42° C. It has been suggested that a subpopulation of IIA NH₂-propeptide trimers that migrated as trimers on SDS-PAGE. In this regard, Fisher et al. reported that the natural type I NH₂-propeptide is not efficiently denatured by SDS treatment prior to electrophoresis. Together, these findings indicate the synthesis of a stable, trimeric IIA NH₂-propeptide nearly identical to that found in vivo.

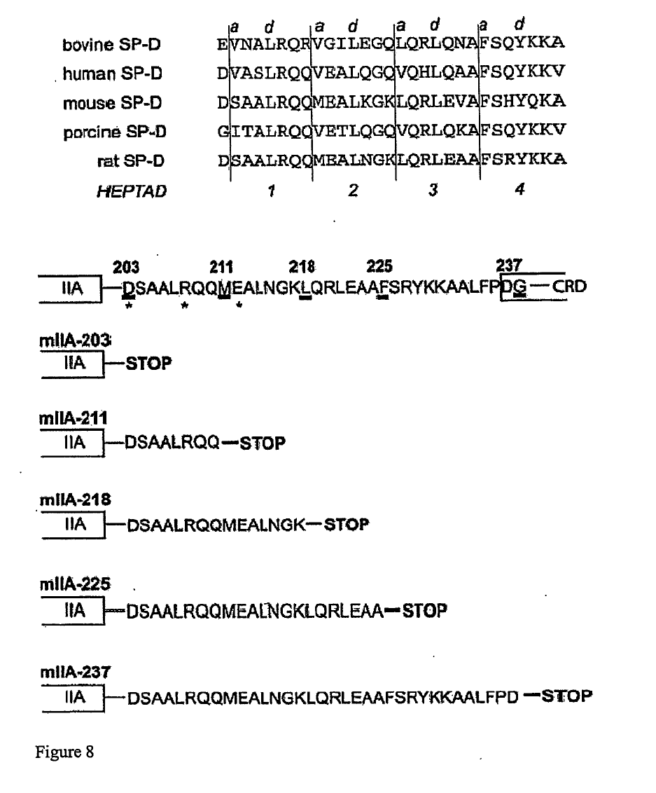

The ability of a 14 amino acid sequence to direct trimerization is surprising. Previous studies have shown that a classical two heptad repeat coiled-coil sequence is unable to form an autonomous folding unit (Su et al., Biochemistry 33:15501-15510 (1994)). Even the complete neck domain of SP-D is short compared to many coiled-coil domains, which average 7 repeats or 14 helical turns for three-stranded coiled-coils. The potential importance of β-branched side-chains for determining the assembly of coiled-coils was emphasized by Harbury et al. (Science 262:1401-1407 (1993)). In that study the occurrence of β-branched residues at the “d” position disfavored dimers, while these residues at the “a” position disfavored tetramers, and the presence of branched residues at both positions favored trimers. Given the occurrence of valine residues in the first three “a” positions of the human SP-D neck sequence (FIG. 8), it has been suggested that this feature contributes to trimeric assembly.

However, no β-branched amino acids occur in these positions in the rat sequence, SMLRQQMEALNGK (SEQ ID NO:2), and none of the other known SP-Ds or related collectins show a similar conservation of P-branched residues in this position (e.g., bovine SP-D, VNALRQRVGILEGQ, SEQ ID NO:3). Studies using model peptides and surveys of known coiled-coils have identified residues that favor various oligomeric states. Residues found in the “a” and “d” positions of SP-D are usually non-discriminatory with respect to oligomerization or favor dimer formation. For example, leucine, which is present in the “d” position of the first three heptad repeats of SP-D, marginally favors dimers over trimers. Consistent with these observations, analysis of both human and rat (-helical coiled-coil sequences using MultiCoil predicted a dimeric association. For example, dimer formation probability for the human SP-D coiled-coil sequence was approximately 90%, or 70% for the rat sequence, using the available windows of 21 residues.

Thus, it seems likely that other interactions contribute to the stability or oligomerization of the 14 amino acid sequence. In this regard, g-e′ ionic interactions can contribute to the stability and oligomerization of some α-helical coiled-coils. Although most discussions emphasize the effects of electrostatic interactions on stability, Beck et al. recently showed that specific electrostatic interactions were required for trimerization of the considerably longer coiled-coil domain of cartilage matrix protein. Inspection of the neck sequence of rat SP-D suggests the possible occurrence of an intra-helical ionic interaction (i to i+4 spacing between Arg²⁰⁸ and Glu²¹²) and/or an inter-chain ionic interaction (i to i+5 spacing between Asp²⁰³ and Arg²⁰⁸; g-e′) (FIG. 8).

In any case, the finding that mIIA-218 is secreted as monomers, while IIA-218 is secreted as trimers, shows that the collagen domain contributes to trimer stability. Thus, both the amino-terminal heptad repeats of the neck of SPD and the IIA collagen sequence are required to form stable chimeric trimers. This represents the direct demonstration of a cooperative and mutually-stabilizing interaction between a collagen domain and its non-collagenous trimerization domain.

The mIIA-237 fusion protein reproducibly trimerizes, but without a detectable dimeric intermediate. Trimerization was also more efflcient, requiring less cross-linker than for the other truncation mutants. We speculate that this “all-or-none” cross-linking of mIIA-237 results from the presence of two contiguous sites for BS³ cross-linking at Lys²²⁹-Lys²³⁰ within the fourth heptad repeat. Although this seems at odds with the observation that cross-linking of IIA/SP-ID also proceeds through a dimeric intermediate, the three chains may not be within an equivalent environment compared to the context of the intact neck+CRD domain.

The crystal structure of the human SP-D neck+CRD shows a striking deviation from 3-fold symmetry involving the fourth heptad repeat, with one of the three tyrosines at position 228 bured, and the other two partially exposed (Hakansson et al., Structure 7:255-264 (1999)). Thus, our findings are consistent with the possibility that asymmetry is imposed on the neck by the presence of the CRD domain. Another potential implication is that the observed asymmetry exists in solution, and is not simply an artifact of crystallization.

Any three identical or different polypeptides containing the neck-region may form homotrimers or heterotrimers under appropriate conditions. A homotrimer consists of three polypeptides which are the same. A heterotrimer consists of three polypeptides, at least two of which are different. All three polypeptides may be different. One, two or all three polypeptides in a heterotrimer may be a polypeptide according to the invention, provided each polypeptide has a region able to trimerize.

The present invention further provides nucleic acid comprising a sequence of nucleotides encoding a polypeptide able to form a trimer and comprising SEQ ID NO:1, an amino acid sequence variant thereof or derivative thereof, or a sequence of amino acids having an amino acid pattern and/or hydrophobicity profile the same as or similar to SEQ ID NO:1, fused to a heterologous sequence of amino acids, as disclosed herein. The nucleic acid may comprise an appropriate regulatory sequence operably linked to the encoding sequence for expression of the polypeptide. Expression from the encoding sequence may be said to be under the control of the regulatory sequence. Preferably, a variant, derivative or sequence having an amino acid pattern and/or hydrophobicity profile will follow the following formula:

(abcdefg)ₙ

wherein positions a and d are occupied by hydrophobic residues; positions e and g by charged residues; positions b, c and f by polar or charged residues; and n is 2. Although less preferable, a collagenous molecule monomer may comprise two contiguous sites for BS³ cross-linking within the fourth heptad repeat of SP-D, the heptad repeat having the formula:

(abcdefg)ₙ

wherein positions a and d are occupied by hydrophobic residues; positions e and g by charged residues; positions b, c and f by polar or charged residues; and n is 4. In addition, a truncated fusion protein may consist of two heptad repeats of SP-D, the heptad repeat having the formula:

(abcdefg)ₙ

wherein positions a and d are occupied by hydrophobic residues; positions e and g by charged residues; positions b, c and f by polar or charged residues; and n is 2. Finally, a truncated fusion protein consisting of three heptad repeats of SP-D may be provided, the heptad repeat having the formula:

(abcdefg)ₙ

wherein positions a and d are occupied by hydrophobic residues; positions e and g by charged residues; positions b, c and f by polar or charged residues; and n is 3.

Also provided by the present invention are a vector comprising nucleic acid as set out above, particularly any expression vector from which the encoded polypeptide can be expressed under appropriate conditions, and a host cell containing any such vector or nucleic acid.

A convenient way of producing a polypeptide according to the present invention is to express nucleic acid encoding it. Accordingly, the present invention also encompasses a method of making a polypeptide according to the present invention, the method comprising expression from nucleic acid encoding the polypeptide, either in vitro or in vivo. The nucleic acid may be part of an expression vector. Expression may conveniently be achieved by growing a host cell, containing appropriate nucleic acid, under conditions which cause or allow expression of the polypeptide.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include HeLa cells, baby hamster kidney cells and many others. A common, preferred bacterial host is E. coli.

Suitable vectors can be chosen or constructed, containing appropriate regulatory sequences, including. promoter sequences, terminator fragments, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology, Second Edition, Ausubel et al. eds., John Wiley & Sons, 1992. The relevant disclosures of Sambrook et al. and Ausubel et al. are incorporated herein by reference.

Thus, a further aspect of the present invention provides a host cell containing nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g., by culturing host cells under conditions for expression of the gene.

In one embodiment, the nucleic acid of the invention is integrated into the genome (e.g., chromosome) of a host cell. Integration may be promoted by inclusion of sequences which promote recombination with the genome, in accordance with standard techniques. Following expression, polypeptides may be caused or allowed to trimerize. This may be prior to or following isolation.

A method of seeding a collagenous triple-helix involves causing or allowing trimerization of such a polypeptide. It may involve first the production of the polypeptide by expression from encoding nucleic acid therefore. The present invention provides such nucleic acid, a vector comprising such nucleic acid, including an expression vector from which the polypeptide may be expressed, and a host cell transfected with such a vector or nucleic acid. The production of the polypeptide may involve growing a host cell containing nucleic acid encoding the polypeptide under conditions in which the polypeptide is expressed. Systems for cloning and expression, etc. are discussed supra, and are well known in the art.

### EXAMPLES

Without further elaboration, it is believed that one skilled in the art can, using the preceding description, utilize the present invention to its fullest extent. The following specific examples are offered by way of illustration and not by way of limiting the remaining disclosure.

### Example 1

### Purification of IIA/SP-D fusion protein

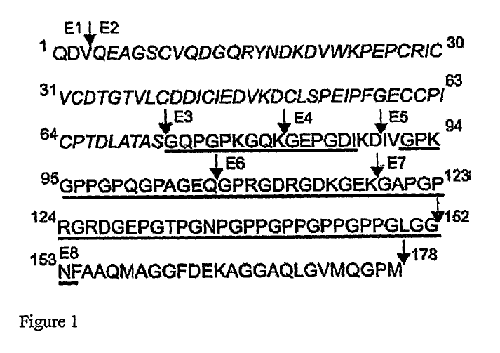

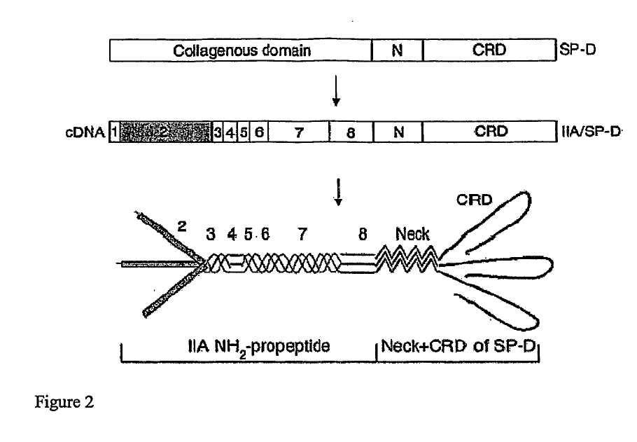

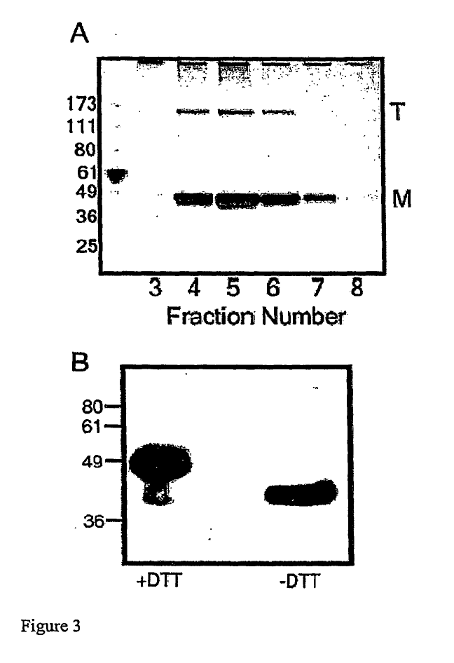

In order to study the polypeptides and trimerization methods of the present invention, a chimeric gene construct was synthesized consisting of cDNA encoding full-length type IIA NH₂-propeptide (exons 1-8; FIG. 1) fused to the cDNA encoding the neck domain and lectin domain of SP-D. The cDNA of SP-D, the chimeric construct and the predicted structure of the resulting fusion protein, named IIA/SP-D, are shown in FIG. 2. IIA/SP-D was efficiently purified from all other contaminating proteins present in the conditioned medium of stably-transfected CHO cells after maltosyl-agarose chromatography (FIG. 3A). The monomer protein showed an apparent molecular weight of 45 kD in the absence of sulfhydryl reduction when compared to globular protein standards used in this gel system. Interestingly, a small population of stable trimers of IIA/SP-D, resistant to SDS treatment and boiling prior to gel electrophoresis, were also visualized. Similar stable trimers of the type I procollagen NH₂-propeptide have been detected from bone (Fisher et al., J. Biol. Chem. 262:13457-13463 (1987)).

Immunoblotting of the EDTA-eluted protein with anti-IIA, anti-IIE 3-8 or anti-SPD polyclonal antisera confirmed identification of IIA/SP-D. Results were identical with all three antibodies and FIG. 3B shows the immunopositive IIA/SP-D bands after detection with the anti-Exon 3-8 antibody. The fusion protein migrated mnore slowly after sulfhydryl reduction due to unfolding of the looped structure created by formnation of the two intra-chain disulfide bonds present within the lectin domain of SP-D. Even though the type IIA NH₂-propeptide domain is predicted to contain five intra-chain disulfide bonds, the loops are comparatively small and disruption of these bonds did not alter the ellectrophoretic migration of the protein (results not shown). However, disruption of the cysteine pairs within the IIA NH₂-propeptide altered the structure of the exon 2-encoded domain such that recognition of the epitope by the anti-IIA antibody was affected (results not shown). All ten cysteine residues in this domain are paired because reaction of IIA/SP-D with Ellman's reagent (Pierce Chemical Co.) showed no quantifiable yellow-colored product as would be expected in the presence of free sulfhydryl groups. This suggests the presence of a very intricately-folded domain since the ten cysteine residues within type IIA NH₂-propepticle are arranged in close proximity to each other (FIG. 1).

### Example 2

### Analysis of the IIA NH₂-Propeptide Collagen Domain

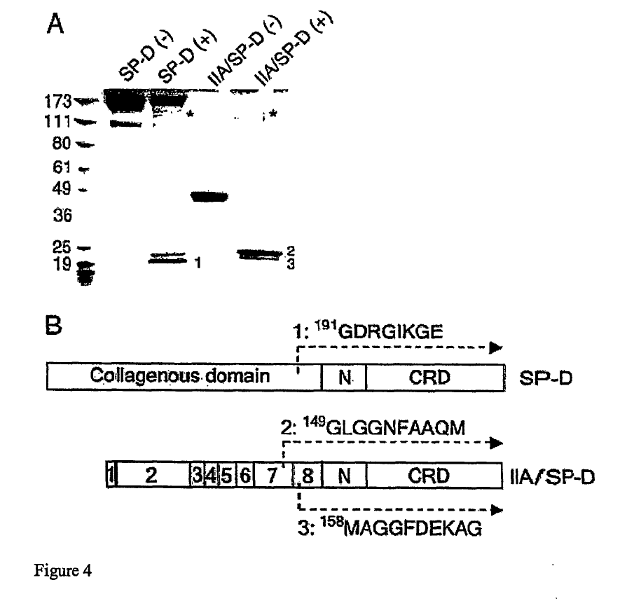

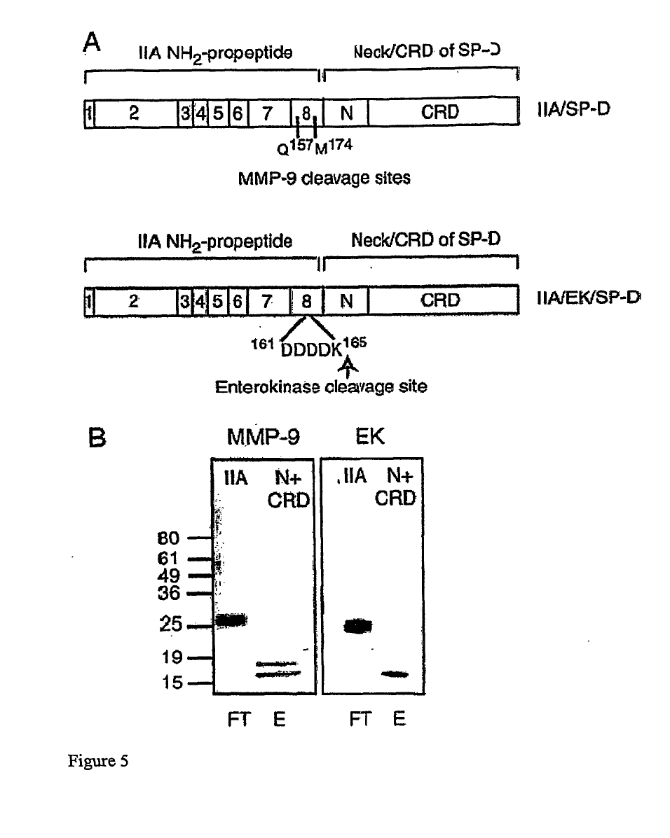

To investigate the structure of the recombinant IIA NH₂-propeptide, IIA/SP-D fusion protein was digested with purified bacterial collagenase, and the major collagenase-resistant bands were characterized by N-terminal sequencing. SP-D, which contains its own collagen domain, was included as a control. As shown in FIG. 4A (protein bands 1,2 and 3), most of the Gly-X-Y collagen domain in IIAISP-D and SP-D was digested (FIG. 4B). In addition, amino acid analysis of IIA/SP-D showed that there were 8 hydroxyproline residues in the collagen domain of IIA NH₂-propeptide. There are 11 potential sites for proline hydroxylation (Gly-X-Pro), but it is not known what percentage of prclines is hydroxylated in the native type II propeptide. To further determine the trimeric configuration of the IIA NH₂-propeptide, we chose to purify the propeptide from the neck/CRD of SP-D. This was done by cleavage of the wild-type IIA/SP-D protein with MMP-9 or by digestion of the mutant fusion protein (IIAIEK/SP-D) synthesized with an enterokinase cleavage site within the exon 8-encoded telopeptide domain (FIG. 5A). After cleavage, the digested protein fragments were applied to a maltosyl-agarose column, which binds to the tnmeric neck/CRD fragments. The IIA NH₂-propeptide was present in the flow-through and the SP-D fragments were then eluted with EDTA (FIG. 5B).

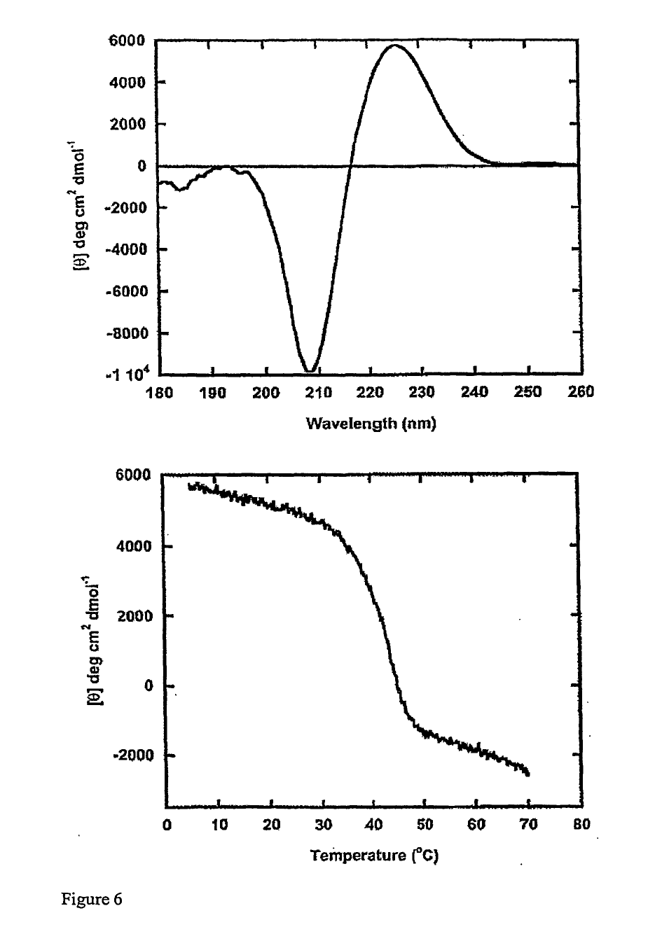

To confirm that the IIA NH₂-propeptide contained a correctly-folded collagen triple helix, the propeptide purified by enterokinase cleavage of IIA/EK/SP-D was analyzed by circular dichroism (CD) spectroscopy. The CD spectrum of a collagen triple helix is characterized by a small positive peak at 220-225 nm, a crossover at 213 nm and a trough at approximately 197 nm (Goodman et al., Biopolymers 47:127-142 (1998)). FIG. 6A shows a large positive ellipticity at 225 nm, indicative of a collagen triple helix. The IIA propeptide was heated to 70° C. and the CD spectrum was monitored at 225 nm. FIG. 6B shows that the mean residue ellipticity (θ) decreased with increasing temperature and that the melting temperature of the collagen triple helix was approximately 42° C. Final confirmation that the IIA propeptide exists as a trimer in solution was achieved by analytical ultracentrifugation, using the sedimentation equilibrium approach, to calculate the molecular weight. The expected molecular weight of the trimeric propeptide was estimated using a ProtParam program (http:flus.expasy.org/tools/protparam.html) and was found to be 50,118 g/mol. The actual molecular weight calculated using the sedimentation equilibrium method was 50,838 g/mol.

### A Trimerization Domain is Necessary for the Production of a Correctly-Folded IIA NH₂-Propeptide

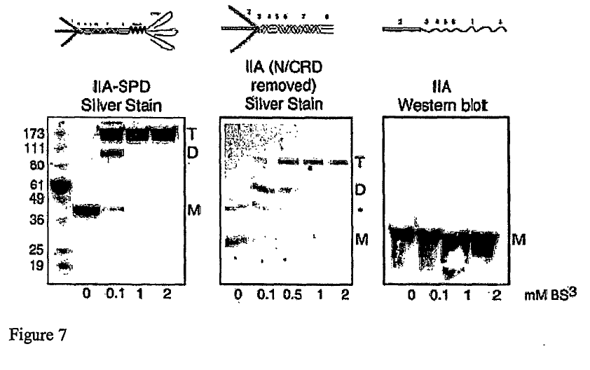

Chemical crosslinking was used to examine the state of oligomerization of the IIA collagen domain. In particular, crosslinking profiles were compared for: 1) the wild-type IIA/SP-D fusion protein, 2) the IIA NH₂-propeptide purified after MMP-9 cleavage of the fusion protein, and 3) the IIA NH₂-propeptide synthesized without fusion to the neck/CRD domains of SP-D. As shown in FIG. 7, crosslinking resulted in the dose-dependent appearance of IIA/SP-D trimers (T) through a dimeric (D) intermediate. As expected, the isolated IIA NH₂-propepbde showed a similar crosslinking pattern. By contrast, the IIA NH₂-propeptide expressed in the absence of SP-D sequence showed no evidence of crosslinked dimers or trimers, indicating the secretion of monomers (M).

### A 14 Amino Acid Sequence of the Coiled-Coil Neck Domain Drives the Trimerization of the IIA NH₂-Propeptide

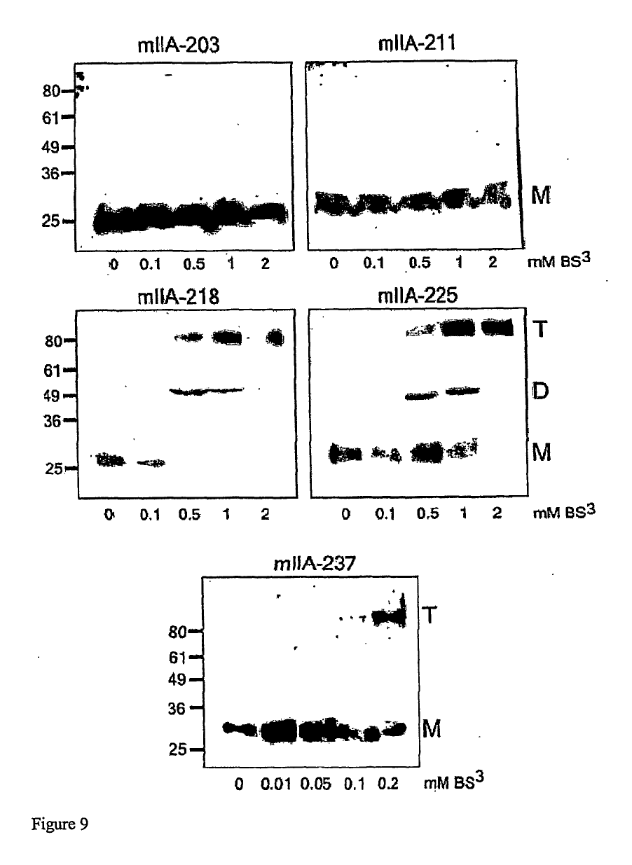

The trimerization domain of rat SP-D is a coiled-coil structure that consists of four heptad repeats as depicted in FIG. 8. In order to further assess the relaUve contributions of sub-regions of the neck domain, IIA/SP-D truncation mutants were synthesized by introducing premature stop codons within the coiled-coil neck domain to produce the IIA NH₂-propeptide attached to one, two, or three contiguous heptad repeats. Two additional mutant IIA/SP-D proteins were generated as controls. One contained a stop codon at the first amino acid of the neck domain (Asp²⁰³) or at the final residue (Gly²³⁷) of the 35 amino acid sequence originally identified as the SP-D trimerization unit (Hoppe et al., FEBS Letters 344:191-195 (1994); Kishore et al., Biochem. J. 318:505-511 (1996)) (FIG. 8). Each mutant protein was covalently cross-linked and the presence of protein monomers, dimers or trimers was detected by immunoblotting using the anti-IIA antibody. FIG. 9 shows that the IIA NH₂-propeptides lacking neck domain sequence (mIIA-203) or fused to the first heptad repeat (mIIA-211) were secreted as monomers. However, truncated fusion proteins containing two or three heptad repeats (mIIA-218 and mIIA-225, respectively), showed trimeric assembly. The IIA NH₂-propeptide attached to the 35 amino acid stretch of the coiled-coil neck (mIIA-237) was also secreted as a non-covalent trimer. However, lower concentrations of cross-linker (0.1-0.2 mM) were sufficient for detection of mIIA-237 trimers compared to concentrations used to detect trimers of the other truncated mutant proteins (0.5-1 mM), and no dimeric intermediate was identified. Co-operativity exists between the IIA NH₂-propeptide collagen domain and the 14 amino acid sequence of the SP-D coiled-coil neck domain

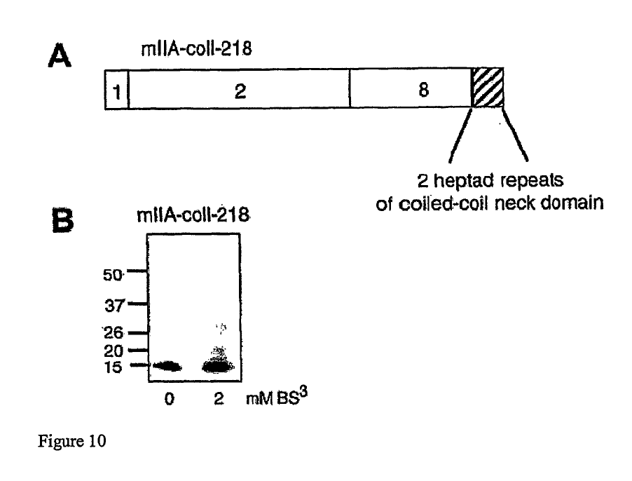

Based on published literature, it was shown that a two heptad repeat coiled-coil sequence cannot form an autonomous folding unit (Su et al., Biochemistry 33:15501-15510 (1994)). Thus, it is highly likely that co-operative interactions exists between the collagen domain and the short, 14 amino acid sequence of the SP-D trimerization domain to stabilize the truncated fusion protein (mIIA-218, FIGS. 8 and 9). A collagen deletion construct was synthesized to produce a mutant protein consisting of exon 1, 2 and 8 of the IIA NH₂-propeptide fused to two heptad repeats of the coiled-coil domain (mIIA-coll-218; FIG. 10A). Protein from conditioned media of transiently-transfected CHO cells was cross-linked with BS³ and detected by SDS-PAGE and Western blotting using the anti-IIA antiserum. FIG. 10B shows that the fusion protein is still monomeric after addition of the highest concentration of cross-linker, confirming the importance of the collagen domain in the stabilization of the protein.

### Example 3

### Expression of IIA/SP-D Fusion Protein in CHO-K1 Cells

A chimeric construct was synthesized by linking the cDNA encoding the NHz propeptide of type IIA procollagen (FIG. 1) to the cDNA encoding the neck+CRD of rat SP-D (FIG. 2). This chimeric construct and resulting fusion protein was named IIA/SP-D. The cDNA encoding exons 1-8 of human type IIA procollagen NH₂-propeptide was amplified by RT-PCR from RNA that had been isolated from articular chondrocytes in culture. Specific upstream and downstream primers were designed from the pro-al type II collagen complete coding sequence (Accession: L10347; SEQ ID NO:4). The IIA/SP-D chimeric construct was made by overlap extension PCR. Briefly, the complete coding sequence of IIA NH₂-propeptide (using oligo A: ggtacgaattcatgattcgcctcggg; SEQ ID NO:5; this primer sequence contains extra bases and the EcoRI site at the 5′ end, shown in bold) and a 3′ sequence homologous to a region of the neck domain of rat SP-D (using oligo B: cagcactgtccattggtccttgcat; SEQ ID NO: 6) was amplified by PCR for 25 cycles at an annealing temperature of 52° C. The same conditions were used to amplify the neck+CRD of rat SP-D containing a 5′ sequence homologous to a region of the IIA cDNA (using oligo C: aggaccaatggacagtgctgctctg; SEQ ID NO: 7) and a 3′-EcoRI site (using a T7-specific downstream oligonucleotide). cDNA products from the two PCR amplifications were combined and overlap extension PCR was carried out for 30 cycles at an annealing temperature of 55° C. using oligos A and T7. The resulting chimeric construct was digested with EcoRI (Promega, Madison, Wisconsin), subcloned into pGEM-3Z (Promega, Madison, Wis.) and the orientation of the subdloned insert was confirmed by restriction mapping and DNA sequencing.

IIA/SP-D cDNA was excised from pGEM-3Z by EcoRI digestion and ligated into the multiple cloning site of a vector suitable for expression of the polypeptide in Chinese Hamster Ovary (CHO) cells (Ausubel et al., Current Protocols in Molecular Biology (Ausubel, R. M., Brent, R., Kingston, R. E., Moore, S. S., Seidman, J. G., Smith, J. A., and Struhl, K., Eds.), John Wiley & Sons, New York (2000)) distal to a cytomegalovirus promoter/enhancer and proximal to a glutamine synthetase gene. CHO cells (CHO-K1; ATCC CCL-61) were transfected with the ligated vector-IIA/SP-D using Lipofectamine (Invitrogen, Carlsbad, Calif.) and grown in selection Glasgow's minimum essential medium (GMEM; Invitrogen, Carlsbad, Calif.) containing 10% dialyzed FBS and the glutamine synthetase inhibitor, methionine sulfoxamine (MSX; 25-50 μM) for 2-3 weeks. Stable clones were obtained as described by Crouch and colleagues for the expression of recombinant rat-SPD (Crouch et al., J. Biol. Chem. 269:15808-15813 (1994)). To assess the importance of the trimerizing neck domain, a control vector construct was constructed consisting only of cDNA encoding full-length IIA NH₂-propeptide, devoid of cDNA encoding the neck and lectin domains. This construct was used in transient transfections of CHO cells using Lipofectamine reagent.

### Detection and Purification of IIA/SP-D Fusion Protein

Media from transiently transfected CHO cells were screened for the presence of the fusion protein by an enzyme linked immunoassay using rabbit anti-human exon 2 (IIA) antibody (Oganesian et al., J. Histo. Cytochem 45:1469-1480 (1997)), chicken IgY anti-human Exon 3-8 antibody or rabbit anti-rat SP-D antibody (Persson et al., J. Biol. Chem. 265:5755-5760 (1990)). Immuno-positive proteins labeled with rabbit-HRP secondary antibodies were detected by enhanced chemiluminescence using SuperSignal® chemiluminescent substrate (Pierce Chemical Co., Rockford, Ill.). Clones expressing the IIA/SP-D fusion protein were selected and cultured further by exposure to 50-100 μM MSX and resulting conditioned media was dialyzed against TBS, pH 7.5, containing 10 mM EDTA. CaCl₂ (20 mM) was added to the dialyzed media and IIA/SP-D was subsequently purified by maltosyl-agarose chromatography (Church et al., supra). Because the interaction of the CRD with maltose is calcium-dependent (Persson et al., supra), IIA/SP-D was eluted from the column with TBS/10 mM EDTA, pH 7.5. Eluted fractions were analyzed by SDS-polyacrylamide gel electrophoresis, silver staining and Western blotting.

### Collagenase Digestion of IIA/SP-D

Bacterial collagenase was purified by gel filtration chromatography using crude collagenase as the starting material (Worthington Biochemical Corp., Lakewood, N.J.) (Peterkofsky et al., Biochemistry 10:988-994 (1971)). IIA/SP-D or rat SP-D (30 pig) in TBS/10 mM EDTA, pH 7.5, was digested with purified bacterial collagenase (1 μg) containing CaCl₂ (20 mM) and N-ethylmaleimide (5 mM), overnight at 37° C. Fresh collagenase (1 μg) was added for a further 3 hours at 37° C. followed by EDTA (4 mM) to stop the reaction. An aliquot (5 μg) of digested and undigested IIA/SP-D or rat SP-D was electrophoresed through a 4-20% SDS-polyacrylamide gel to confirm collagenase digestion. The major collagenase-resistant products were detected by Coomassie blue staining and subjected to N-terminal amino acid sequencing. Collagenase-digested IIA/SP-D or SP-D was transferred to Sequi-Blot PVDF membrane (Bio-Rad, Hercules, Calif.), stained with Coomassie blue, excised and sequenced on an ABI 473A protein sequencer equipped with model 610A data analysis software.

### Purification of IIA NH₂-Propeptide: MMP-9 or Enterokinase Cleavage of Wild-Type or Mutant IIA/SP-D Fusion Protein

Approximately 100 μg of wild-type IIA/SP-D fusion protein was digested overnight at 37° C. with human recombinant MMP-9 at an enzyme:substrate ratio of 1:100. MMP-9 cleaves within the telopeptide domain of the IIA propeptide on either side of Q¹⁵⁷ and M¹⁷⁴ (Persson et al., supra). Since MMP-9 has two cleavage sites within the telopeptide and cleavage is not always 100% efficient, we proceeded to synthesize a mutant IIA/SP-D chimeric construct containing an enterokinase cleavage site in the exon 8-encoded telopeptide. Using the QuikChangerm Site-Directed Mutagenesis Kit (Stratagene, La Jolla, Calif.), oligonucleotide primers were designed to change the DNA sequence encoding amino acids 161-165 in exon 8 of the wild-type IIA NH₂-propeptide (¹⁵¹GFDEK¹⁸⁵) to one which encodes the EK cleavage site (¹¹⁶DDDDK¹⁶⁵). Stable CHO cell lines producing this mutant fusion protein (IIA/EK/SP-D) were produced as described above. Approximately 0.001% w/w of enterokinase (New England Biolabs, Beverly, Mass.) was added to purified IIA/EK/SP-D protein overnight at room temperature.

Cleavage by MMP-9 or enterokinase was confirmed by gel electrophoresis, silver staining and immunoblotting using antibodies specific for the IIA (exon 2) domain or the CRD of SP-D. Cleaved products were calcified and applied to a maltosyl-agarose column to separate the IIA NH₂-propeptide (present in the flow-through) from the neck/CRD of SP-D (present in the EDTA eluate).

### Chemical Cross-Linking

Covalent cross-linking was performed using bis-(sulfosuccinimidyl)suberate (BS³; Pierce Chemical Co., Rockford, Ill.). Increasing amounts of BS³(0, 0.1, 0.5, 1 and 2 mM final concentration) prepared in 5 mM sodium citrate, pH 5, was added to each recombinant proteins for 1 hour at room temperature. Addition of SDS-PAGE loading buffer containing Tris-HCl (0.5 M) inhibited the reaction. Samples were boiled for 5 minutes prior to SDS-PAGE, which was carried out in the absence of sulfhydryl reduction. Cross-linked proteins were identified by silver staining or immunolocalization using anti-IIA (exon 2) polyclonal antisera.

### Circular Dichroism and Determination of IIA NH₂-Propeptide Melting Temperature

Approximately 50 μg of IIA NH₂-propeptide (0.2 mg/ml in PBS, pH 7.5), purified by cleavage of the mutant IIA/EK/SP-D fusion protein containing the enterokinase cleavage site, was analyzed by circular dichroism (CD) spectroscopy. A Jasco (Easton, Md.) J715 spectropolarimeter with a thermostated quartz cell, path length of 0.1 cm, was used and the spectrum was recorded at 5° C. between 180-260 nm. To determine the melting temperature of the IIA NH₂-propeptide, the spectrum was monitored at 225 nm from 5° C. to 70° C.

### Analytical Ultracentrifugation

Equilibrium sedimentation experiments were performed using a Beckman (Fullerton, Calif.) Optima XL-A analytical ultracentrifuge using a six-channel centerpiece in an AN-60 Ti rotor. IIA NH₂-propeptide, purified from enterokinase cleavage of the IIA/EK/SP-D mutant protein, in PBS (pH 7.5) was analyzed at three concentrations: 0.2, 0.4 and 0.8 mg/ml. Experiments were performed at two speeds (20,000 and 28,000 rpm) at a temperature of 20° C. and wavelength of 280 nm. Data were fitted using WinNonlin® (Pharsight, Mountain View, Calif.) V1.035

(http://www.ucc.uconn.edu/˜wwwbiotc/UAF.html) and a partial specific volume of 0.73 cm³/g was used for determining the molecular weight.

### Synthesis of IIA/SP-D Mutant Constructs Containing a Premature Termination Codon in the Coiled-Coil Neck Domain

To determine the minimum sequence that can function as a trimerizing unit, mutant constructs were designed containing termination codons at specific locations within the heptad repeats of the coiled-coil. Using IIA/SP-D cDNA in pGEM-3Z as a substrate, four mutant constructs were synthesized using the QuikChangem Site-Directed Mutagenesis Kit (Stratagene, La Jolla, Calif.). The sequence of the mutant was confirmed by DNA sequencing. Mutant IIA/SP-D cDNA constructs were excised from pGEM-3Z by EcoRI digestion and sub-cloned into a vector suitable for expression of the polypeptide in CHO cells. Correct orientation of the mutant cDNA insert in the vector was confirmed by restriction enzyme digestion (Hindlil and Bglll, Promega, Madison, Wis.) and agarose gel electrophoresis. CHO cells were transiently-transfected with each mutant construct using FuGENE 6 reagent (Roche, Switzerland) according to the manufacturer's instructions. Proteins were precipitated from the conditioned medium overnight at 4° C. with 33% ammonium sulfate. Precipitated proteins were washed three times in saturated ammonium sulfate, resuspended in PBS and dialyzed overnight in cold PBS. Chemical cross-linking of each mutant protein was carried out as described above. Proteins were detected by SDS-PAGE and immunolocalization of Western blots using the anti-IIA polyclonal antibody.

### Synthesis of a Collagen Deletion Mutant Construct

To determine if the minor collagen domain of the IIA NH₂-propeptide contributes to the stability of the truncated fusion protein, we generated a related truncation mutant with an associated deletion of the collagen sequence (mIIA-218, FIG. 8). One pair of oligonucleotide primers was designed to amplify exons 1 and 2 of the IIA NH₂-propeptide (upstream oligo A: ggtacgaattcatgattcgcctcggggct (SEQ ID NO: 8); downstream oligo B: taaaggatccaactttgctgcccag (SEQ ID NO: 9)). Another pair was designed to amplify exon 8 to the 3′ CRD region of SP-D (upstream oligo C: aatggatccaactgctgcccag (SEQ ID NO: 10); downstream oligo D: gtaccgaattctcagaactcacag (SEQ ID NO: 11)). BamHI site is shown in bold. Two separate PCRs were done using the mutant chimeric cDNA construct containing a premature stop codon at the end of the second heptad repeat of SP-D (mIIA-218; FIG. 8) as a substrate. A cDNA fragment (approximately 300 bp) was amplified using oligos A and B for 30 cycles (95° C. for 30s, 55° C. for 30s, 72° C. for 30s) and another cDNA fragment (approximately 650 bp) was amplified using oligos C and D for 30 cycles (95° C. for 30s, 55° C. for 30s, 72° C. for 1 min 30s). Each DNA fragment was digested with BamHI, ligated together, and another round of PCR was done, using oligos A and D, for 30 cycles (95° C. for 30s, 55° C. for 30s, 72° C. for 2 min) to amplify the ligated fragment. The resulting cDNA fragment devoid of exons 3-7 encoding the collagen domain of the IIA NH₂-propeptide, was cloned into a vector suitable for expression of the polypeptide in CHO cells. Orientation of the cloned insert was confirmed by restriction mapping and DNA sequencing. CHO cells were transiently-transfected with the collagen deletion mutant construct using FuGENE 6 reagent (Roche, Switzerland) according to the manufacturer's instructions. Proteins were precipitated from the conditioned medium overnight at 4° C. with 33% ammonium sulfate. Precipitated proteins were washed three times in saturated ammonium sulfate, resuspended in PBS and dialyzed overnight in cold PBS. The collagen deletion mutant protein (mIIA-coll-218) was cross-linked using BS³ and detected by SDS-PAGE and Western blotting using the anti-IIA polyclonal antibody.

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### References Cited

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention. Specifically referred to and included herein in its entirety is a publication by K McAlinden, et a/., entitled: Trimerizatfon of the amino propeptfde of type IIA procollagen using a 14-amino acid sequence derived from the coiled-coil neck domain of surfactant protein D. J Biol Chem. 277(43):41274-81 (2002).

1-35. (canceled)

36. A method for trimerizing polypeptides, the method comprising:
(a) providing at least three polypeptides comprising a collagenous domain linked to a non-collagenous trimerization domain, said trimerization domain having the formula
(abcdefg)ₙ
wherein positions a and d comprise hydrophobic residues; positions e and g comprise charged residues; positions b, c and f comprise polar or charged residues; n is 2 or 3; and
(b) contacting the polypeptides.

37. A method according to claim 36, wherein the non-collagenous trimerization domain comprises an amino acid sequence corresponding to the first two or three heptad repeats of a neck domain of a mammalian pulmonary surfactant protein D.

38. A method according to claim 37, wherein the mammalian pulmonary surfactant protein D is rat pulmonary surfactant protein D.

39. A method according to claim 37, wherein the mammalian pulmonary surfactant protein D is human pulmonary surfactant protein D.

40. A method according to claim 36, wherein the trimerization domain amino acid sequence is selected from the group consisting of SEQ ID NOs: 1 to 10.

41. A polypeptide trimer comprising three polypeptides comprising a collagenous domain linked to a non-collagenous trimerization domain, said trimerization domain having the formula
(abcdefg)ₙ
wherein positions a and d comprise hydrophobic residues; positions e and g comprise charged residues; positions b, c and f comprise polar or charged residues; and n is 2 or 3.

42. A polypeptide trimer according to claim 41, wherein the non-collagenous trimerization domain comprises an amino acid sequence corresponding to the first two or three heptad repeats of a neck domain of a mammalian pulmonary surfactant protein D.

43. A polypeptide trimer according to claim 42, wherein the mammalian pulmonary surfactant protein D is rat pulmonary surfactant protein D.

44. A polypeptide trimer according to claim 42, wherein the mammalian pulmonary surfactant protein D is human pulmonary surfactant protein D.

45. A polypeptide trimer according to claim 41, wherein the trimerization domain amino acid sequence is selected from the group consisting of SEQ ID NOs: 1 to 10.

46. A polypeptide trimer according to claim 41, wherein the trimer is a homotrimer.

47. A polypeptide trimer according to claim 42, wherein the trimer is a heterotrimer.

48. A polypeptide comprising a collagenous domain linked to a non-collagenous trimerization domain, said trimerization domain having the formula
(abcdefg)ₙ
wherein positions a and d comprise hydrophobic residues; positions e and g comprise charged residues; positions b, c and f comprise polar or charged residues; and n is 2 or 3.

49. A polypeptide according to claim 48, wherein the non-collagenous trimerization domain comprises an amino acid sequence corresponding to the first two or three heptad repeats of a neck domain of a mammalian pulmonary surfactant protein D.

50. A polypeptide according to claim 49, wherein the mammalian pulmonary surfactant protein D is rat pulmonary surfactant protein D.

51. A polypeptide according to claim 49, wherein the mammalian pulmonary surfactant protein D is human pulmonary surfactant protein D.

52. A polypeptide according to claim 48, wherein the trimerization domain amino acid sequence is selected from the group consisting of SEQ ID NOs: 1 to 10.

53. A polynucleotide comprising a nucleic acid sequence encoding the polypeptide of claim 48.

54. A polynucleotide according to claim 53 comprised by a vector, wherein the vector comprises the polynucleotide operably linked to a regulatory nucleic acid sequence capable of initiating expression of the polypeptide.

55. A mammalian cell containing a polypeptide trimer according to claim 41.

56. A mammalian cell containing a polypeptide according to claim 48.

57. A mammalian cell containing a polynucleotide according to claim 53.
